(19) United States
(12) Patent Application Publication
(10) Pub. No.: US 2021/0093591 A1
(43) Pub. Date: Apr. 1, 2021

(54) METHODS FOR MITIGATING AND PREVENTING PROTEOSTASIS-BASED INJURIES

(71) Applicants: San Diego State University (SDSU) Foundation, dba San Diego State University Research Foundation, San Diego, CA (US); THE SCRIPPS RESEARCH INSTITUTE, La Jolla, CA (US)

(72) Inventors: Christopher C. GLEMBOTSKI, San Diego, CA (US); Erik Alexander BLACKWOOD, San Diego, CA (US); Jeff KELLY, San Diego, CA (US); Rockland WISEMAN, San Diego, CA (US)

(21) Appl. No.: 17/043,966

(22) PCT Filed: Mar. 29, 2019
(86) PCT No.: PCT/US2019/024783

§ 371 Date: Sep. 30, 2020

Related U.S. Application Data
(60) Provisional application No. 62/754,801, filed on Nov. 2, 2018.
(60) Provisional application No. 62/651,029, filed on Mar. 30, 2018.

(51) Int. Cl. A61K 31/167; A61P 9/10

(52) U.S. Cl. CPC A61K 31/167; A61P 9/10

(57) ABSTRACT

Provided are pharmaceutical compositions, formulations, products of manufacture and kits, and methods, for: mitigating, ameliorating, treating or preventing a proteostasis-based injury; selectively inducing only the ATF6 arm of the unfolded protein response in a cell, a tissue or in a mammal, wherein optionally the mammal is a human; protecting a mammalian heart or a mammalian tissue from an acute or a long term ischemia/reperfusion injury or damage; pharmacologically activating ATF6 or the ATF6 arm of the unfolded protein response in a cell or in vivo; comprising: administering to the cell, the tissue, the mammal or the individual in need thereof: (a) a compound as provided herein, for example, the exemplary compound 147.

### RELATED APPLICATIONS

This Patent Convention Treaty (PCT) International Application claims the benefit of priority under 35 U.S.C. § 119(e) of U.S. Provisional Patent Application Ser. No. 62/651,029, Mar. 30, 2018; and, U.S. Ser. No. 62/754,801 filed Nov. 2, 2018. The aforementioned applications are expressly incorporated herein by reference in its entirety and for all purposes. All publications, patents, patent applications cited herein are hereby expressly incorporated by reference for all purposes.

### STATEMENT AS TO FEDERALLY SPONSORED RESEARCH

This invention was made with government support under grant nos. R01 HL75573, R01 HL104535, P01 HL085577, R01 DK102635, R01 DK107604, R01 NS092829, and UL1TR001114. The government has certain rights in the invention.

### TECHNICAL FIELD

This invention generally relates to medicine. In alternative embodiments, provided are pharmaceutical compositions, formulations, products of manufacture and kits, and methods, for: mitigating, ameliorating, treating or preventing a proteostasis-based injury (including e.g., an ischemia/reperfusion (I/R) injury); selectively inducing only the ATF6 arm of the unfolded protein response (UPR) in a cell, a tissue or in a mammal, wherein optionally the mammal is a human; protecting a mammalian heart or a mammalian tissue from an acute or a long term ischemia/reperfusion (I/R) injury or damage, wherein optionally the tissue is a brain, a kidney or a liver, and optionally the heart or tissue is a human heart or tissue; pharmacologically activating ATF6 or the ATF6 arm of the unfolded protein response (UPR) in a cell or in vivo; ameliorating, preventing or treating the loss of cardiac myocytes during ischemia/reperfusion (I/R) injury or damage, ameliorating, preventing or treating ischemic heart disease in an individual in need thereof; and/or ameliorating, preventing or treating acute myocardial infarction (AMI) or tissue loss or damage occurring as a result of the AMI in an individual in need thereof, comprising: administering to the cell, the tissue, the mammal or the individual in need thereof: (a) a compound as provided herein, for example, the exemplary compound 147, or a pharmaceutically acceptable salt or solvate thereof, or optical isomer thereof, or racemic mixture or enantiomer thereof, or (b) a pharmaceutical composition or formulation comprising a compound of (a).

### BACKGROUND

Protein homeostasis, or proteostasis is maintained by pathways that coordinate protein synthesis and folding with the degradation of misfolded, potentially toxic proteins^{1,2}. ER proteostasis is particularly important, since nearly one-third of all proteins are made and folded in the ER, then transported to their final destinations as integral membrane or soluble secreted proteins³. Imbalances in proteostasis cause or exacerbate numerous pathologies, spawning interest in the exogenous manipulation of proteostasis as a therapeutic approach for such diseases⁴. ER proteostasis is regulated by the unfolded protein response (UPR), a stress-responsive signaling pathway comprising three sensors/effectors of ER protein misfolding; PERK (protein kinase R [PKR]-like ER kinase), IRE1 (inositol requiring enzyme 1), and ATF6 (activating transcription factor 6)⁵. Considerable evidence supports ATF6, a transcriptional regulator of ER proteostasis, as a viable therapeutic target for exogenous manipulation of proteostasis⁶⁻¹¹; however, such an approach has not been examined in vivo.

Ischemic heart disease is the leading cause of human deaths worldwide¹². These deaths are mainly due to acute myocardial infarction (AMI), where thrombotic coronary artery occlusion causes rapid, irreparable ischemic injury to the heart, increasing susceptibility to progressive cardiac degeneration and eventual heart failure¹³⁻¹⁵. The treatment of choice for AMI is primary percutaneous coronary intervention, or coronary angioplasty¹⁴, which results in reperfusion. While reperfusion limits ischemic injury, the reperfusion itself injures the heart, in part by increasing reactive oxygen species (ROS). ROS contribute to AMI injury, also known as ischemia/reperfusion (I/R) injury, mainly by damaging proteins, which impairs proteostasis^{16,17}. In fact, reperfusion accounts for up to 50% of the final damage from AMI¹⁸; however, there is no clinically available intervention that mitigates reperfusion injury at the time of coronary angioplasty, underscoring the importance of developing therapies that reduce ROS during reperfusion¹⁴.

Using a mouse model of global ATF6 deletion, we recently showed that, in the heart, ATF6 is responsible for the expression of a broad spectrum of genes not traditionally identified to be regulated by ATF6, including many antioxidant genes that could improve proteostasis during I/R²⁰. There have been no reports addressing whether the unfolded protein response (UPR), including ATF6 as one of the three sensors/effectors of ER protein misfolding in the UPR, can be pharmacologically activated and shown to beneficial in any animal model of pathology.

### SUMMARY

In alternative embodiments, provided are methods for:
selectively inducing only the ATF6 arm of the unfolded protein response (UPR) in a cell, a tissue or in a mammal, wherein optionally the mammal is a human,
mitigating, ameliorating, treating or preventing a proteostasis-based injury or dysfunction, wherein optionally the proteostasis-based injury or dysfunction comprises an ischemia/reperfusion (I/R) injury or damage in any tissue or organ (e.g., heart, kidney, liver, muscle, central nervous system, or brain), or a dysregulated proteostasis in the liver, and optionally the heart or tissue is a human heart or tissue,
protecting a mammalian heart or a mammalian tissue from an acute or a long term ischemia/reperfusion (I/R) injury or damage, wherein optionally the tissue is a brain, a kidney or a liver, and optionally the heart or tissue is a human heart or tissue,
pharmacologically activating ATF6 or the ATF6 arm of the unfolded protein response (UPR) in a cell or in vivo,
ameliorating, preventing or treating the loss of cardiac myocytes during ischemia/reperfusion (I/R) injury or damage,
ameliorating, preventing or treating ischemic heart disease in an individual in need thereof,
ameliorating, preventing or treating acute myocardial infarction (AMI) or tissue loss or damage occurring as a result of the AMI in an individual in need thereof, and/or
ameliorating, preventing or treating an amyloid-based disease, optionally amyloidosis, or an amyloid-based or amyloid-related neurodegenerative disease, wherein optionally the amyloid-based or amyloid-related neurodegenerative disease is a central nervous system (CNS) or peripheral nervous system (PNS) neurodegenerative disease, optionally Alzheimer's disease,

comprising:

administering to the cell, the tissue, the mammal or the individual in need thereof:

(a) (i) a compound have a structure a set forth in Formula I:

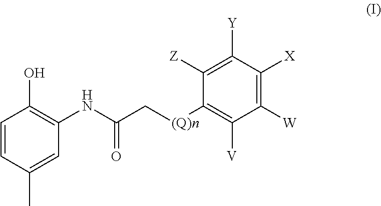

wherein Q is S, O, CH₂, CHF, or CF₂, n=1, 2, 3, or 4, when Q is CH₂, CHF, or CF₂; n is 1 when Q is S or O, and V, W, X, Y and Z are each independently hydrogen, halogen, alkyl, alkenyl, alkynyl, or alkoxy; or a pharmaceutically acceptable salt thereof,

wherein optionally the compound having a structure a set forth in Formula I is compound 147:

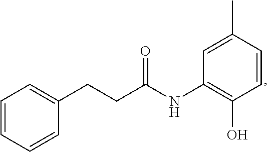 [SMILES: Cc1ccc(O)c(NC(=O)CCc2ccccc2)c1]

(ii) a pharmaceutically acceptable salt or solvate, an optical isomer, or a racemic mixture or enantiomer of a compound of (i),

(iii) a compound as set forth in WO2017/117430 A1, or a pharmaceutically acceptable salt or solvate, optical isomer, or racemic mixture or enantiomer thereof;

(iv) a compound as set forth in FIGS. 7 to 12, or a pharmaceutically acceptable salt or solvate, optical isomer, or racemic mixture or enantiomer thereof; or,

(v) any mixture of compounds of (i) to (v); or

(b) a pharmaceutical composition or formulation comprising a compound of (a), or comprising at least one compound of (a), and optionally further comprising a pharmaceutically acceptable excipient,

thereby:
selectively inducing only the ATF6 arm of the unfolded protein response (UPR) in a cell, a tissue or in a mammal, wherein optionally the mammal is a human,
mitigating, ameliorating, treating or preventing a proteostasis-based injury or dysfunction, wherein optionally the proteostasis-based injury or dysfunction comprises an ischemia/reperfusion (I/R) injury or damage in any tissue or organ (e.g., heart, kidney, liver, muscle, central nervous system, or brain), or a dysregulated proteostasis in the liver, and optionally the heart or tissue is a human heart or tissue,
protecting a mammalian heart, kidney, liver or brain, or a mammalian tissue from an acute or a long term ischemia/reperfusion (I/R) injury or damage, wherein optionally the tissue is a brain, a kidney or a liver, and optionally the heart or tissue is a human heart or tissue,
pharmacologically activating ATF6 or the ATF6 arm of the unfolded protein response (UPR) in a cell or in vivo,
ameliorating, preventing or treating the loss of cardiac myocytes during ischemia/reperfusion (I/R) injury or damage,
ameliorating, preventing or treating ischemic heart disease in an individual in need thereof,
ameliorating, preventing or treating acute myocardial infarction (AMI) or tissue loss or damage occurring as a result of the AMI in an individual in need thereof, and/or
ameliorating, preventing or treating an amyloid-based disease, optionally amyloidosis, or an amyloid-based or amyloid-related neurodegenerative disease, wherein optionally the amyloid-based or amyloid-related neurodegenerative disease is a central nervous system (CNS) or peripheral nervous system (PNS) neurodegenerative disease, optionally Alzheimer's disease.

In alternative embodiments of methods as provided herein,

the compound, pharmaceutical composition or formulation is administered in the form of an implant or a stent, wherein optionally the implant or stent has contained therein or carries, releases or delivers the compound, pharmaceutical composition or formulation, thereby delivering or contacting the compound, pharmaceutical composition or formulation to or with the cell, the tissue, the mammal or the individual in need thereof;

the compound, pharmaceutical composition or formulation is suitable for or is formulated for: topical, intradermal, oral, parenteral, intrathecal or intravenous (IV) infusion administration, wherein optionally the compound, pharmaceutical composition or formulation is suitable for (or formulated for) administration as a (or in the form of a) patch, adhesive tape, gel, liquid or suspension, powder, spray, aerosol, lyophilate, lozenge, pill, geltab, tablet, capsule, stent and/or implant (e.g., administered via an implant);

the compound, pharmaceutical composition or formulation is suitable for or is formulated for: human or veterinary administration, wherein optionally said composition is suitable for (or formulated for) administration to a domestic, zoo, laboratory or farm animal, and optionally the animal is a dog or a cat; or

the compound, pharmaceutical composition or formulation is administered in a pharmaceutically effective dosage or amount, and optionally the pharmaceutically effective dosage or amount is (or total daily dosage is) between about 0.5 mg and about 5000 mg, between about 1 mg and about 1000 mg; or is between about 5 mg and about 500 mg, 10 mg and about 400 mg, 20 mg and about 250 mg; or is about 5 mg and about 150 mg; or is between about 1 mg and about 75 mg; or is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, or about 75 mg,

and optionally the pharmaceutically effective dosage or amount is administered daily, twice a day (bid), three times a day (tid) or four or more times a day.

In alternative embodiments, provided are products of manufacture comprising or having contained therein a compound, pharmaceutical composition or formulation as provided herein, wherein optionally the product of manufacture is an implant or a stent. In alternative embodiments, the compound, pharmaceutical composition or formulation is delivered in a controlled time-released regimen, e.g., comprising use of a time-release formulation or an implant.

In alternative embodiments, provided are uses of a product of manufacture as provided herein, or a compound, pharmaceutical composition or formulation as provided herein, for:
selectively inducing only the ATF6 arm of the unfolded protein response (UPR) in a cell, a tissue or in a mammal, wherein optionally the mammal is a human,
mitigating, ameliorating, treating or preventing a proteostasis-based injury or dysfunction, wherein optionally the proteostasis-based injury or dysfunction comprises an ischemia/reperfusion (I/R) injury or damage in any tissue or organ (e.g., heart, kidney, liver, muscle, central nervous system, or brain), or a dysregulated proteostasis in the liver, and optionally the heart or tissue is a human heart or tissue,
protecting a mammalian heart, kidney, liver or brain, or a mammalian tissue from an acute or a long term ischemia/reperfusion (I/R) injury or damage, wherein optionally the tissue is a brain, a kidney or a liver, and optionally the heart or tissue is a human heart or tissue,
pharmacologically activating ATF6 or the ATF6 arm of the unfolded protein response (UPR) in a cell or in vivo,
ameliorating, preventing or treating the loss of cardiac myocytes during ischemia/reperfusion (I/R) injury or damage,
ameliorating, preventing or treating ischemic heart disease in an individual in need thereof,
ameliorating, preventing or treating acute myocardial infarction (AMI) or tissue loss or damage occurring as a result of the AMI in an individual in need thereof, and/or
ameliorating, preventing or treating an amyloid-based disease, optionally amyloidosis, or an amyloid-based or amyloid-related neurodegenerative disease, wherein optionally the amyloid-based or amyloid-related neurodegenerative disease is a central nervous system (CNS) or peripheral nervous system (PNS) neurodegenerative disease, optionally Alzheimer's disease.

In alternative embodiments, provided are products of manufacture as provided herein, or a compound, pharmaceutical composition or formulation as provided herein, for use in:
selectively inducing only the ATF6 arm of the unfolded protein response (UPR) in a cell, a tissue or in a mammal, wherein optionally the mammal is a human,
mitigating, ameliorating, treating or preventing a proteostasis-based injury or dysfunction, wherein optionally the proteostasis-based injury or dysfunction comprises an ischemia/reperfusion (I/R) injury or damage in any tissue or organ (e.g., heart, kidney, liver, muscle, central nervous system, or brain), or a dysregulated proteostasis in the liver, and optionally the heart or tissue is a human heart or tissue,
protecting a mammalian heart, kidney, liver or brain, or a mammalian tissue from an acute or a long term ischemia/reperfusion (I/R) injury or damage, wherein optionally the tissue is a brain, a kidney or a liver, and optionally the heart or tissue is a human heart or tissue,
pharmacologically activating ATF6 or the ATF6 arm of the unfolded protein response (UPR) in a cell or in vivo,
ameliorating, preventing or treating the loss of cardiac myocytes during ischemia/reperfusion (I/R) injury or damage,
ameliorating, preventing or treating ischemic heart disease in an individual in need thereof,
ameliorating, preventing or treating acute myocardial infarction (AMI) or tissue loss or damage occurring as a result of the AMI in an individual in need thereof, and/or
ameliorating, preventing or treating an amyloid-based disease, optionally amyloidosis, or an amyloid-based or amyloid-related neurodegenerative disease, wherein optionally the amyloid-based or amyloid-related neurodegenerative disease is a central nervous system (CNS) or peripheral nervous system (PNS) neurodegenerative disease, optionally Alzheimer's disease.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### DESCRIPTION OF DRAWINGS

The drawings set forth herein are illustrative of exemplary embodiments provided herein and are not meant to limit the scope of the invention as encompassed by the claims.

FIG. 1A-K illustrate data showing that ATF6 in cardiac myocytes protects the heart from I/R injury:

FIG. 1A schematically illustrates how I/R dysregulates proteostasis, leading to activation of all three arms of the unfolded protein response (UPR), and that the ATF6 arm induces genes that adaptively reprogram proteostasis, decrease myocyte death and provide cardioprotection from I/R damage;

FIG. 1B illustrates an image of cardiac myocytes adjacent to an infarct, where the myocytes in the border zone (FIG. 1B, outlined in red, or in the lower half of the image) are exposed to sub-lethal I/R and mount protective stress responses such as the UPR, while the remote region (FIG. 1B, outlined in blue, or in the upper half of the image) is relatively unaffected;

FIG. 1C illustrates an image of a post-acute myocardial infarction (AMI) cross section of the left ventricle of a mouse heart, showing that in response to acute myocardial infarction (AMI), wild type (WT) mice exhibited a robust activation of ATF6, as evidenced by induction of the ATF6 target genes, Grp78 and Cat in the border zone of hearts subjected to acute I/R;

FIG. 1C-D illustrate images of immunohistochemical staining of GRP78 or CAT (cyan), tropomyosin (red), and nuclei (TOPRO-3) in the border zone of wild-type (WT) (FIG. 1C) or ATF6 conditional knockout mouse (ATF6 cKO) (FIG. 1D) hearts subjected to either sham or acute I/R surgery;

FIG. 1E-G graphically illustrate data from quantitative real-time PCR (qPCR) for Grp78 or Cat in sham or border zone of post-I/R hearts in WT (FIG. 1E), ATF6 cKO (FIG. 1F), or in ventricular explants from control or ischemic heart failure patients (FIG. 1G);

FIG. 1H-I graphically illustrate data showing infarct sizes (FIG. 1H) and plasma cardiac troponin I (cTnI) (FIG. 11) in WT and ATF6 cKO mice post-I/R;

FIG. 1J-K graphically illustrate data showing left ventricular developed pressure (LVDP) (FIG. 1J) and relative infarct sizes (FIG. 1K) post-ex vivo I/R; as further discussed in Example 1, below.

FIG. 2A-J illustrate data showing that exemplary compound 147 selectively activates ATF6 in the heart:

FIG. 2A schematically illustrates a diagram of hypothetical mechanism of ATF6 activation by exemplary compound 147;

FIG. 2B schematically illustrates the chemical structure of a synthetic control compound and the exemplary compound 147;

FIG. 2C illustrates an image of an immunoblot of ATF6 and GAPDH in NRVM 24-hours after treatment with compound 147 or TM in fully-reducing condition (lanes 1-6) or non-reducing conditions (lanes 7-12);

FIG. 2D illustrates an immuno-cyto-fluorescence (ICF) image of ATF6 (green), alpha-actinin (red) and nuclei (TOPRO-3) in NRVM 24-hours after treatment with compound 147;

FIG. 2E graphically illustrates chromatin immunoprecipitation (ChIP-qPCR) of known ATF6 target promoter binding elements (ERSE) for Grp78 (hspa5), cat, and negative control targets Heme oxygenase 1 (ho-1) and gapdh NRVM infected with AdV encoding Flag-ATF6 (1-670) 24-hours after treatment with compound 147;

FIG. 2F illustrates an immuno-cyto-fluorescence (ICF) image of GRP78 and CAT (green), alpha-actinin (red) and nuclei (TOPRO-3) in AMVM 24-hours after treatment with compound 147;

FIG. 2G-H graphically illustrate qPCR for Grp78 or Cat in LV of WT (FIG. 2G) or ATF6 cKO (FIG. 2G) hearts 24-hours post-treatment with control or compound 147;

FIG. 2I-J illustrate images of IHC staining of GRP78 or CAT (cyan), tropomyosin (red), and nuclei (TOPRO-3) in left ventricle (LV) of WT (FIG. 2I) or ATF6 cKO (FIG. 2J) hearts 24-hours post-treatment with control or compound 147;

as further discussed in Example 1, below.

FIG. 3A-I illustrate how the exemplary compound 147 improves proteostasis and decreases oxidative stress in an ATF6-dependent manner:

FIG. 3A-B graphically illustrate data from studies where NRVM were infected with AdV-HA-T-cell antigen receptor alpha-chain (TCRα; an ER-transmembrane protein that is chronically misfolded and degraded by ERAD), treated with siCon or siAtf6 and either control or compound 147 for 24-hours prior to cyclohexamide for 0, 0.5 or 1 h; densitometry of the HA-TCRα immunoblots at the respective times (a) and ERAD at the 0.5-hour time point (b) are shown;

FIG. 3C graphically illustrates data from studies where secretory proteostasis assayed in NRVM when transfected with Gaussia luciferase and treated with siCon or siAtf6, and either control or compound 147 for 24-hours; medium was collected and luciferase activity was measured;

FIG. 3D graphically illustrates data from studies where NRVM were transfected with siCon or siAtf6, then treated with or without TM, control or compound 147 for 24 h, after which viability was determined;

FIG. 3E-F graphically illustrate data from studies where NRVM were transfected with siCon or siAtf6, treated with or without control or compound 147 for 24 h, then I/R, after which viability (FIG. 3E) and MDA (FIG. 3F) were measured;

FIG. 3G graphically illustrates data from studies showing the viability of I/R-treated cultured adult cardiomyocytes isolated from WT or ATF6 cKO mice 24-hours post-treatment with control or compound 147;

FIG. 3H-I graphically illustrate data from studies where LVDP (FIG. 3H) and relative infarct sizes (FIG. 3I) of WT or ATF6 cKO mice treated 24 h with control or compound 147 then ex vivo I/R;

as further discussed in Example 1, below.

FIG. 4A-E illustrate how the exemplary compound 147 gene induction timecourse, in vivo:

FIG. 4A schematically illustrates an exemplary experimental design testing the effects of compound 147 in WT untreated mice;

FIG. 4B-C graphically illustrate data from qPCR for Grp78 (b) or Cat (c) in LV of mice from indicated trials;

FIG. 4D schematically illustrates the percent increase in fractional shortening;

FIG. 4E illustrates images of IHC staining of GRP78 or CAT (cyan), tropomyosin (red), and nuclei (TOPRO-3) in LV of mice from respective trials;

as further discussed in Example 1, below.

FIG. 5A-I illustrate how the exemplary compound 147 improves cardiac performance 7 d post-AMI:

FIG. 5A schematically illustrates an experimental design and dosing protocols for animal trials during remodeling phase of AMI;

FIG. 5B and FIG. F-G graphically illustrate echocardiographic parameters of fractional shortening (FIG. 5B), LV end diastolic volume (LVEDV) (FIG. 5F) and LV end systolic volume (LVESV) (FIG. 5G);

FIG. 5C graphically illustrates the ratio of heart weight to body weight;

FIG. 5D graphically illustrates plasma cTnI;

FIG. 5E graphically illustrates diastolic function as determined by pulse wave Doppler (PW) technique to analyze E and A waves;

FIG. 5H-I graphically illustrate qPCR for Grp78 (FIG. 5H) or Cat (FIG. 5I) in LV of mice from indicated trials at culmination of study;

as further discussed in Example 1, below.

FIG. 6A-K illustrate how the exemplary compound 147 exerts widespread protection in multiple organ systems:

FIG. 6A-B graphically illustrate qPCR for Grp78 (FIG. 6A) or Cat (FIG. 6B) in left ventricular, liver, kidney, and brain extracts from WT mice 24-hours post-treatment with control or compound 147;

FIG. 6C graphically illustrates the ratio of transcript levels of Xbp1s to Xbp1 as determined by qPCR in liver extracts from WT or ATF6 KO mice 24-hours post-treatment with control or compound 147 and then treated with 2 mg/kg of TM for designated periods of time.

FIG. 6D graphically illustrates Triglyceride levels in liver extracts from WT or ATF6 KO mice 24-hours post-treatment with control or compound 147 and then treated with 2 mg/kg of TM for 12-hours.

FIG. 6E graphically illustrates preclinical experimental design testing protective effects of compound 147.

FIG. 6F-H illustrate images of relative infarct sizes in the heart (FIG. 6F), kidney (FIG. 6G), and brain (FIG. 6H) of male mice 24 h after reperfusion.

FIG. 6I-K graphically illustrate plasma cTnI (FIG. 6I), plasma creatinine (FIG. 6J), and neurological score (FIG. 6K) based on the Bederson system of behavioral patterns post-cerebral ischemic injury of male mice 24 h after reperfusion of respective injury models;

as further discussed in Example 1, below.

FIG. 7-12 illustrate exemplary compounds used in methods as provided herein:

FIG. 7 illustrates a genus of compounds used in methods as provided herein as exemplified by the illustrated Formula I;

FIG. 8 illustrates a genus of compounds used in methods as provided herein as exemplified by the illustrated Formula II;

FIG. 9 illustrates a genus of compounds used in methods as provided herein as exemplified by the illustrated Formula III;

FIG. 10 illustrates a genus of compounds used in methods as provided herein as exemplified by the illustrated Formula IX;

FIG. 11 illustrates a genus of compounds used in methods as provided herein as exemplified by the illustrated Formula VII;

FIG. 12 illustrates two genuses of compounds used in methods as provided herein as exemplified by the illustrated Formula IV and Formula V.

FIG. 13A-G illustrates how I/R activates the UPR:

FIG. 13A illustrates an image of immunoblots of neonatal rat ventricular myocytes (NRVM) for the proteins shown after I/R or tunicamycin (TM);

FIG. 13B-D graphically illustrate quantification of immunoblots from NRVM subjected to normoxia or I/R; ATF6 (FIG. 13B), IRE1 (FIG. 13C), and PERK (FIG. 13D) activation are displayed as ratios of active fragment ATF6 (50 kd), spliced-XBP1 and phospho-PERK relative to ATF6 (90 kd), IRE1, and PERK, respectively;

FIG. 13E illustrates an image of immune-cytofluorescence (ICF) for GRP78 or CAT (green), alpha-actinin (red) and nuclei (TOPRO-3) in isolated adult cardio-myocytes (AMVM) post-I/R;

FIG. 13F-G graphically illustrate quantification of immunoblots for Grp78 (FIG. 13F) or Cat (FIG. 13G) from NRVM subjected to normoxia or I/R;

as further discussed in Example 1, below.

FIG. 14A-J illustrate that endogenous ATF6 is cardioprotective in a model of a chronic AMI:

FIG. 14A graphically illustrates data from a qPCR for atf6 in isolated adult mouse ventricular myocytes (AMVM), isolated cardiac fibroblasts, or liver extracts from WT or ATF6 cKO mice;

FIG. 14B illustrates: upper image shows an immunoblot for Atf6 and loading control, β-actin, and IHC staining for ATF6 (cyan), tropomyosin (red), and nuclei (TOPRO-3) in LV of WT or ATF6 cKO mice, and lower image shows stained LVs;

FIG. 14C-D graphically illustrate data from a qPCR for IRE1 downstream target, Erdj4, or PERK downstream target, Atf4 in the border zone of WT (FIG. 14C) or ATF6 cKO (FIG. 14D) hearts 24-hours after I/R;

FIG. 14E graphically illustrate the amount of malondialdehyde (MDA) in WT and ATF6 cKO mice 24-hours post-I/R;

FIG. 14F-J graphically illustrate parameters from mice 7-days post I/R; FIG. 14F shows Fractional shortening; FIG. 14G shows ratio of heart weight to body weight; FIG. 14H shows plasma cTnI; FIG. 14I-J show qPCR for Grp78 (FIG. 14I) or Cat (FIG. 14J) in border zone of mice;

as further discussed in Example 1, below.

FIG. 15A-F illustrate data showing that the exemplary compound 147 selectively activates ATF6;

FIG. 15A illustrates an image of an immunoblot of UPR target proteins from NRVM 24-hours after treatment with exemplary compound 147 or tunicamycin (TM);

FIG. 15B-F graphically illustrate quantification of immunoblots of NRVM treated with control or exemplary compound 147;

FIG. 15G illustrates an image of an immunoblot of NRVM infected with AdV encoding Flag-ATF6 (1-670) 24-hours after treatment with control or exemplary compound 147;

FIG. 15H illustrates an image of an immunoblot of UPR target proteins from LV of WT or ATF6 cKO hearts 24-hours after treatment with control or exemplary compound 147;

FIG. 15I-J graphically illustrate data of a qPCR for Erdj4 or Atf4 in LV of WT (FIG. 15I) or ATF6 cKO (FIG. 15J) hearts 24-hours after treatment with control or compound 147;

as further discussed in Example 1, below.

FIG. 16A-F graphically illustrate data showing that exemplary compound 147 exhibits no deleterious effects in vivo:

FIG. 16A-C graphically illustrate data from a qPCR for Erdj4 (FIG. 16A), Atf4 (FIG. 16B), and Atp2a2 (FIG. 16C);

FIG. 16D-F graphically illustrate data of: Ratio of heart weight to body weight (FIG. 16D); Plasma cTnI (FIG. 16E); and, a qPCR for cardiac pathology genes (FIG. 16F), using Nppa (black), Nppb (red), Col1a2 (blue), and Myh7 (green);

as further discussed in Example 1, below.

FIG. 17A-E illustrates that the exemplary compound 147 decreases pathological remodeling 7 d post-AMI:

FIG. 17A-B graphically illustrate data from a qPCR for Erdj4 (FIG. 17A) or Atf4 (FIG. 17B) in border zone of mice;

FIG. 17C illustrates an image of an IHC staining for GRP78 or CAT (cyan), tropomyosin (red), and nuclei (TOPRO-3) in left ventricular free wall of sham hearts or the border zone of hearts;

FIG. 17D graphically illustrate data from a qPCR for cardiac pathology genes: Nppa (black), Nppb (red), Col1a2 (blue), and Myh7 (green) in border zones of mice;

FIG. 17E illustrates an image of an IHC staining for cleaved caspase-3 (cyan), tropomyosin (red), and nuclei (TOPRO-3) in LV free wall of sham hearts or the border zone of hearts;

as further discussed in Example 1, below.

FIG. 18A-H illustrate data showing that exemplary compound 147 is protective in multiple models of myocardial damage:

FIG. 18A illustrates representative images of TTC-stained post-I/R hearts from Trials 8-10 of the acute I/R protocol shown in FIG. 6E;

FIG. 18B-C graphically illustrate data showing the relative infarct sizes (FIG. 18B) and plasma cTnI (FIG. 18C) of female mice 24-hours after reperfusion when following the acute I/R protocol shown in FIG. 6E;

FIG. 18D-E graphically illustrate data showing the relative infarct sizes (FIG. 18D) and plasma cTnI (FIG. 18E) of ATF6 cKO mice 24-hours post-I/R when following experimental Trials 8 (Con) and 9 (exemplary compound 147) of the acute I/R protocol;

FIG. 18F schematically illustrates an exemplary experimental design for testing the effects of exemplary compound 147 in a different model of acute myocardial infarction (AMI) using isoproterenol;

FIG. 18G-H graphically illustrate data showing the relative infarct sizes (FIG. 18G), and plasma cTnI (FIG. 18G);

as further discussed in Example 1, below.

FIG. 19 illustrates supplementary Table 1, showing 7 day I/R echocardiographic parameters, as further discussed in Example 1.

FIG. 20 illustrates supplementary Table 2, showing cardiac performance in Trial 2, as further discussed in Example 1.

FIG. 21 illustrates supplementary Table 3, showing the effects of exemplary compound 147 for 7 days as acute myocardial infarction (AMI) echocardiographic parameters, as further discussed in Example 1.

FIG. 22 illustrates supplementary Table 4, showing the effects of exemplary compound 147 for 7 days as acute myocardial infarction (AMI) echocardiographic parameters, as further discussed in Example 1.

Like reference symbols in the various drawings indicate like elements.

### DETAILED DESCRIPTION

In alternative embodiments, provided are pharmaceutical compositions, formulations, products of manufacture and kits, and methods, for: selectively inducing only the ATF6 arm of the unfolded protein response (UPR) in a cell, a tissue or in a mammal, wherein optionally the mammal is a human; protecting a mammalian heart or a mammalian tissue from an acute or a long term ischemia/reperfusion (I/R) injury or damage, wherein optionally the tissue is a brain, a kidney or a liver, and optionally the heart or tissue is a human heart or tissue; pharmacologically activating ATF6 or the ATF6 arm of the unfolded protein response (UPR) in a cell or in vivo; ameliorating, preventing or treating the loss of cardiac myocytes during ischemia/reperfusion (I/R) injury or damage, ameliorating, preventing or treating ischemic heart disease in an individual in need thereof; and/or ameliorating, preventing or treating acute myocardial infarction (AMI) or tissue loss or damage occurring as a result of the AMI in an individual in need thereof.

As discussed in Example 1, below, we determined that treatment with a pharmacological activator of ATF6 could reprogram proteostasis and mitigate (e.g., treat, ameliorate or prevent) a pathology in a mouse model of ischemic diseases, such as those that affect the heart, e.g., ischemia/reperfusion (I/R) injury or damage.

In alternative embodiments, compound 147 is or comprises a compound having the formula:

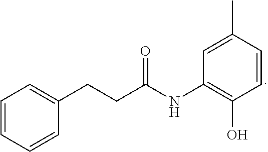 [SMILES: Cc1ccc(O)c(NC(=O)CCc2ccccc2)c1]

In alternative embodiments, provided are pharmaceutical compositions, dosage forms or formulations having contained therein, or comprising: (a) a compound 147, or a pharmaceutically acceptable salt or solvate thereof, or optical isomer thereof, or racemic mixture or enantiomer thereof; or, (b) a pharmaceutical composition or formulation comprising a compound of (a).

In alternative embodiments, the pharmaceutical compositions, dosage forms or formulations as provided herein are suitable for or formulated are for: topical, oral, parenteral, intrathecal or intravenous infusion administration, wherein optionally said composition is suitable for (or formulated for) administration as a (or in the form of a) patch, adhesive tape, gel, liquid or suspension, powder, spray, aerosol, lyophilate, lozenge, pill, geltab, tablet, capsule, stent and/or implant. The pharmaceutical composition, dosage form or formulation can be suitable for or is formulated for human or veterinary administration, wherein optionally said composition is suitable for (or formulated for) administration to a domestic, zoo, laboratory or farm animal.

In alternative embodiments, for compounds used to practice methods as provided herein, all single enantiomer, diastereomeric, and racemic forms of a structure are intended. Compounds used in methods as provided herein can include enriched or resolved optical isomers at any or all asymmetric atoms as are apparent from the depictions, at any degree of enrichment. Both racemic and diastereomeric mixtures, as well as the individual optical isomers can be isolated or synthesized so as to be substantially free of their enantiomeric or diastereomeric partners, and any of these can be used to practice embodiments as provided herein.

In alternative embodiments, when a group is recited, the group can be present in more than a single orientation within a structure resulting in more than single molecular structure, e.g., a carboxamide group C(═O)NR, it is understood that the group can be present in any possible orientation, e.g., X—C(═O)N(R)—Y or X—N(R)C(═O)—Y, unless the context dearly limits the orientation of the group within the molecular structure.

In alternative embodiments, an alkyl group has no limitations on the number of atoms in the group. refers to a saturated chain containing only carbon atoms, which may be linear or branched. In alternative embodiments, alkyl groups can be substituted at any atom independently with additional alkyl, alkyloxy, alkylamino, alkylthio, acyl, sulfonyl, cycloalkyl, heterocycloalkyl, phenyl or heteroaryl groups as defined herein. Alkyl groups may be substituted at any atom independently with heteroatoms chosen from N, O or S, which may be further substituted independently with additional hydrogen, alkyl, alkyloxy, alkylamino, alkylthio, acyl, sulfonyl, cycloalkyl, heterocycloalkyl, phenyl or heteroaryl groups. When the heteroatom is N, it may be substituted twice independently with hydrogen, alkyl, alkyloxy, alkylamino, alkylthio, acyl, sulfonyl, cycloalkyl, heterocycloalkyl, phenyl or heteroaryl groups. When the heteroatom is N, O or S, it may form a double bond to the chain as in a ketone or an oxime. When the heteroatom is S, it may be oxidized at S with one or more O atoms, as a sulfoxide or sulfone. Alkyl groups may be substituted at any atom independently with halogens chosen from F, Cl, Br or I, and may be disubstituted as in, for example, a —CF₂— group in the chain, or trisubstituted as in, for example, a —CF₃ group at the terminus of the chain. Alkyl groups may be fused through a single disubstituted atom in the chain to a ring to form a cycloalkyl or heterocycloalkyl structure. Alkyl group size is defined, for example, as C₁₋₆, which refers to the number of atoms in the group. Some non-limitative examples of linear alkyl groups include methyl, ethyl, propyl, butyl, pentyl or hexyl. Some non-limitative examples of branched alkyl groups include isopropyl, isobutyl, sec-butyl, tert-butyl or tert-amyl.

In alternative embodiments, alkyl groups include straight chain and branched carbon-based groups having from 1 to about 20 carbon atoms, and typically from 1 to 12 carbons or, in some embodiments, from 1 to 8 carbon atoms, or from 1 to 4 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoaikyl groups as well as other branched chain forms of alkyl. Representative substituted alkyl groups can be substituted one or more times with any of the substituent groups listed above, for example, amino, hydroxy, cyano, carboxy, nitro, thio, alkoxy, and halogen groups.

In alternative embodiments, cycloalkyl groups are groups containing one or more carbocyclic ring including, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above.

In alternative embodiments, alkenyl groups include straight and branched chain and cyclic alkyl groups as defined above, except that at least one double bond exists between two carbon atoms. Thus, alkenyl groups have from 2 to about 20 carbon atoms, and typically from 2 to 12 carbons or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others. Exemplary alkenyl groups include, but are not limited to, a straight or branched group of 2-8 or 3-4 carbon atoms. Exemplary alkenyl groups include, but are not limited to, vinyl, allyl, butenyl, pentenyl, and the like. In alternative embodiments, the term “alkenyl” refers to a fully or partially unsaturated chain containing only carbon atoms, which may be linear or branched, containing at least one carbon-carbon double bond. Alkenyl groups may be further substituted at any atom independently with additional alkyl, alkyloxy, alkylamino, alkylthio, acyl, sulfonyl, cycloalkyl, heterocycloalkyl, phenyl or heteroaryl groups as defined herein. Alkenyl groups may be substituted at any atom independently with heteroatoms chosen from N, O or S, which may be further substituted independently with additional alkyl, alkyloxy, alkylamino, alkylthio, acyl, sulfonyl, cycloalkyl, heterocycloalkyl, phenyl or heteroaryl groups. When the heteroatom is N, it may be substituted twice independently with alkyl, alkyloxy, alkylamino, alkylthio, acyl, sulfonyl, cycloalkyl, heterocycloalkyl, phenyl or heteroaryl groups.

In alternative embodiments, the term “substituted” refers to an organic group as defined herein in which one or more bonds to a hydrogen atom contained therein are replaced by one or more bonds to a non-hydrogen atom such as, but not limited to, a halogen (e.g., F, CI, Br, or I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitnies, nitro groups, nitroso groups, N-oxides, hydrazides, azides, and enamines; and other heteroatoms in various other groups. Non-limiting examples of substituents that can be bonded to a substituted carbon (or other) atom include F, CI, Br, I, OR, CN, NO, NO₂, ONO₂, azido, CF₃, OCF₃, R, O (oxo), S (thiono), methylenedioxy, ethylenedioxy, N(R)₂, SR, SOR, SO₂R, SO₂IM(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)O₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂₎ N(COR)COR, N(OR)R, C(═NH)N(R)₂, C(O)N(OR)R, or C(═NOR)R wherein R can be hydrogen or a carbon-based moiety, and wherein the carbon-based moiety can itself be further substituted; for example, R can be hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl can be further independently mono- or multi-substituted with the substituent, or with some or all of the above-listed functional groups, or with other functional groups; or wherein two R groups bonded to a nitrogen atom or to adjacent nitrogen atoms can together with the nitrogen atom or atoms form a heterocyclyl, which can be further mono- or independently multi-substituted with the substituent, or with some or all of the above-listed functional groups, or with other functional groups.

In alternative embodiments, cycloalkenyl groups include cycloalkyl groups having at least one double bond between 2 carbons. Thus for example, cycloalkenyl groups include but are not limited to cyclohexenyl, cyclopentenyl, and cyclohexadienyl groups. Cycloalkenyl groups can have from 3 to about 8-12 ring members, whereas in other embodiments the number of ring carbon atoms range from 3 to 5, 6, or 7. Cycloalkyl groups can further include polycyclic cycloalkyl groups such as, but not limited to, norbornyl, adamantyl, bornyl, camphenyl, isocamphenyl, and carenyl groups, and fused rings such as, but not limited to, decalinyl, and the like, provided they include at least one double bond within a ring. Cycloalkenyl groups also can include rings that are substituted with straight or branched chain alkyl groups as defined above.

In alternative embodiments, alkynyl groups include straight and branched chain alkyl groups, except that at least one triple bond exists between two carbon atoms. In alternative embodiments, alkynyl groups have from 2 to about 20 carbon atoms, and typically from 2 to 12 carbons or, in some embodiments, from 2 to 8 carbon atoms. Examples include, but are not limited to ˜—C≡CH, —C≡C(CH₃), —C≡C(CH₂CH₃), —CH₂C≡CH₃, —CH₂C≡C(CH₃), and —CH₂C≡C(CH₂CH₃) among others. In alternative embodiments, the term “alkynyl” refers to a fully or partially unsaturated chain containing only carbon atoms, which may be linear or branched, containing at least one carbon-carbon triple bond. Alkynyl groups may be further substituted at any atom independently with additional alkyl, alkyloxy, alkylamino, alkylthio, acyl, sulfonyl, cycloalkyl, heterocycloalkyl, phenyl or heteroaryl groups as defined herein. Alkynyl groups may be substituted at any atom independently with heteroatoms chosen from N, O or S, which may be further substituted independently with additional alkyl, alkyloxy, alkylamino, alkylthio, acyl, sulfonyl, cycloalkyl, heterocycloalkyl, phenyl or heteroaryl groups. When the heteroatom is N, it may be substituted twice independently with alkyl, alkyloxy, alkylamino, alkylthio, acyl, sulfonyl, cycloalkyl, heterocycloalkyl, phenyl or heteroaryl groups.

In alternative embodiments, aryl groups are cyclic aromatic hydrocarbons that do not contain heteroatoms in the ring. An aromatic compound, as is well-known in the art, can be a multiply-unsaturated cyclic system that contains 4n+2 π electrons where n is an integer. Thus aryl groups can include, but are not limited to, phenyl, azulenyl, heptaienyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined above. Representative substituted Aryl groups can be mono-substituted or substituted more than once, such as, but not limited to, 2-, 3-, 4-, 5-, or 8-substituted phenyl or 2-8 substituted naphthyl groups, which can be substituted with carbon or non-carbon groups such as those listed above.

In alternative embodiments, heterocyclyl groups or the term “heterocyclyl” includes aromatic and non-aromatic ring compounds containing 3 or more ring members, of which one or more ring atom is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members.

In alternative embodiments, heteroaryl groups are heterocyclic aromatic ring compounds containing 5 or more ring members, of which, one or more is a heteroatom such as, but not limited to, N, O, and S; for instance, heteroaryl rings can have 5 to about 8-12 ring members. In alternative embodiments, a heteroaryl group is a variety of a heterocyclyl group that possesses an aromatic electronic structure, which is a multiply-unsaturated cyclic system that contains 4n+2 π electrons wherein n is an integer.

In alternative embodiments, the term “alkoxy” or “alkoxyl” refers to an oxygen atom connected to an alkyl group, including a cycloalkyl group, as are defined above. Examples of linear alkoxy groups include but are not limited to methoxy, ethoxy, n-propoxy, n-butoxy, n-pentyloxy, n-hexyloxy, and the like. Examples of branched alkoxy include but are not limited to isopropoxy, sec-butoxy, tert-butoxy, isopentyloxy, isohexyloxy, and the like. Exemplary alkoxy groups include, but are not limited to, alkoxy groups of 1-6 or 2-8 carbon atoms, referred to herein as Ci-ealkoxy, and Ca-ealkoxy, respectively. Exemplary alkoxy groups include, but are not limited to methoxy, ethoxy, isopropoxy, etc.

In alternative embodiments, the terms “halo” or “halogen” or “halide” by themselves or as part of another substituent mean, unless otherwise stated, a fluorine, chlorine, bromine, or iodine atom, preferably, fluorine, chlorine, or bromine.

In alternative embodiments, a “haloalkyl” group includes mono-halo alkyl groups, poly-halo alkyl groups wherein ail halo atoms can be the same or different, and per-halo alkyl groups, wherein ail hydrogen atoms are replaced by the same or differing halogen atoms, such as fluorine and/or chlorine atoms. Examples of haloalkyl include trifluoromethyl, 1,1-dichloroethyl, 1,2-dichloroethyl, 1,3-dibromo-3,3-difluoropropyl, perfluorobutyl, and the like.

In alternative embodiments, the term “phenyl” refers to a benzene ring, which may be substituted independently at any position with additional hydrogen, alkyl, alkyloxy, alkylamino, alkylthio, acyl, sulfonyl, cyano, cycloalkyl, heterocycloalkyl, phenyl or heteroaryl groups as defined herein. Phenyl groups may be substituted at any atom independently with heteroatoms chosen from N, O or S, which may be further substituted independently with additional hydrogen, alkyl, alkyloxy, alkylamino, alkylthio, acyl, sulfonyl, cycloalkyl, heterocycloalkyl, phenyl or heteroaryl groups. When the heteroatom is N, it may be substituted twice independently with hydrogen, alkyl, alkyloxy, alkylamino, alkylthio, acyl, sulfonyl, cycloalkyl, heterocycloalkyl, phenyl or heteroaryl groups. When the heteroatom is S, it may be oxidized at S with one or more O atoms, as a sulfoxide or sulfone. Phenyl groups may be substituted at any atom independently with halogens chosen from F, Cl, Br or I. Phenyl groups may be fused through two adjacent atoms to an additional ring, which may be substituted cycloalkyl, heterocycloalkyl, phenyl or heteroaryl as defined herein. Phenyl groups may optionally include multiple ring fusions, optionally further substituted with spirocyclic fusions or bridged structures, or a combination of these, as part of a larger ring system containing multiple rings. Some non-limitative examples of phenyl groups include benzene, naphthalene, indane or tetrahydronaphthalene.

Standard abbreviations for chemical groups such as are well known in the art are used; e.g., e=methyl, Et=ethyl, i-Pr=isopropyl, Bu=butyl, t-Bu=tert-butyl, Ph=phenyl, Bn=benzyl, Ac=acetyl, Bz=benzoyl, and the like.

In alternative embodiments, a “pharmaceutically acceptable” or “pharmacologically acceptable” salt is a salt formed from an ion that has been approved for human consumption and is generally nontoxic, such as a chloride salt or a sodium salt.

If a value of a variable that is necessarily an integer, e.g. , the number of carbon atoms in an alkyl group or the number of substituents on a ring, is described as a range, e.g., 0-4, what is meant is that the value can be any integer between 0 and 4 inclusive, i.e. , 0, 1 , 2, 3, or 4.

The compounds described herein used to practice methods as provided herein can be prepared in a number of ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials. For example, commercially available chemicals can be obtained from Aldrich, Alfa Aesare, Wako, Acros, Fisher, Fiuka, Maybridge or the like and can be used without further purification, except where noted. Dry solvents are obtained, for example, by passing these through activated alumina columns.

The compounds and intermediates as used in methods as provided herein be isolated from their reaction mixtures and purified by standard techniques such as filtration, liquid-liquid extraction, solid phase extraction, distillation, re-crystallization or chromatography, including flash column chromatography, or HPLC.

### Bioisosteres of Compounds

In alternative embodiments, compounds used in methods as provided herein, e.g., (a) compound 147, or a pharmaceutically acceptable salt or solvate thereof, or optical isomer thereof, or racemic mixture or enantiomer thereof; or, (b) a pharmaceutical composition or formulation comprising a compound of (a), include or comprise their respective bioisosteres. In alternative embodiments, bioisosteres used to practice methods as provided herein comprise one or more substituent and/or group replacements with a substituent and/or group having substantially similar physical or chemical properties which produce substantially similar biological properties to a compound, or stereoisomer, racemer or isomer thereof. In one embodiment, the purpose of exchanging one bioisostere for another is to enhance the desired biological or physical properties of a compound without making significant changes in chemical structures.

For example, in one embodiment, bioisosteres of compounds used to practice methods as provided herein, or used in products of manufacture as provided herein, are made by replacing one or more hydrogen atom(s) with one or more fluorine atom(s), e.g., at a site of metabolic oxidation; this may prevent metabolism (catabolism) from taking place. Because the fluorine atom is similar in size to the hydrogen atom the overall topology of the molecule is not significantly affected, leaving the desired biological activity unaffected. However, with a blocked pathway for metabolism, the molecule may have a longer half-life or be less toxic, and the like.

### Formulations and Pharmaceutical Compositions

In alternative embodiments, provided are compounds and compositions, including formulations and pharmaceutical compositions, for use in in vivo, in vitro or ex vivo methods for practicing methods as provided herein. In alternative embodiments, provided are pharmaceutical compositions, and methods of making and using them, for e.g., mitigating, ameliorating, treating or preventing a proteostasis-based injury (including e.g., an ischemia/reperfusion (I/R) injury); selectively inducing only the ATF6 arm of the unfolded protein response (UPR) in a cell, a tissue or in a mammal, wherein optionally the mammal is a human; protecting a mammalian heart or a mammalian tissue from an acute or a long term ischemia/reperfusion (I/R) injury or damage, wherein optionally the tissue is a brain, a kidney or a liver, and optionally the heart or tissue is a human heart or tissue; pharmacologically activating ATF6 or the ATF6 arm of the unfolded protein response (UPR) in a cell or in vivo.

In alternative embodiments, the pharmaceutical compositions as provided herein can be administered parenterally, topically, orally or by local administration, such as by aerosol or transdermally. In alternative embodiments, pharmaceutical compositions can be prepared in various forms, such as granules, tablets, pills, capsules, suspensions, taken orally, suppositories and salves, lotions and the like. Pharmaceutical formulations as provided herein may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, lozenges, gels, geltabs, on patches, in implants, etc. In practicing embodiments as provided herein, the pharmaceutical compounds can be delivered by transdermally, by a topical route, formulated as applicator sticks, solutions, suspensions, emulsions, gels, creams, ointments, pastes, jellies, paints, powders, and aerosols. Oral carriers can be elixirs, syrups, capsules, tablets, pills, geltabs and the like.

In alternative embodiments, provided are pharmaceutically acceptable salts of compounds as provided herein, including pharmaceutically acceptable non-toxic bases or acids including inorganic or organic bases and inorganic or organic acids. In alternative embodiments, salts are derived from inorganic bases such as aluminum, ammonium, calcium, copper, ferric, ferrous, lithium, magnesium, manganic salts, manganese, potassium, sodium, zinc, and the like; or, salts can be in a solid form, or in a crystal structure, or the form of hydrates. In alternative embodiments, salts are pharmaceutically acceptable organic non-toxic bases including salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. In alternative embodiments, e.g., if a compound provided herein is basic, salts are prepared from pharmaceutically acceptable non-toxic acids, including inorganic and organic acids. Such acids include acetic, benzenesulfonic, benzoic, camphorsulfonic, carbonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like.

In alternative embodiments, pharmaceutically acceptable salts include hemisalts of non-toxic acids or bases, or hemihydrates.

In alternative embodiments, compounds and compositions used to practice methods as provided herein are delivered orally, e.g., as pharmaceutical formulations for oral administration, and can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients can be carbohydrate or protein fillers, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxymethyl cellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate.

In alternative embodiments, liquid carriers are used to manufacture or formulate compounds as provided herein, or a composition used to practice the methods as provided herein, including carriers for preparing solutions, suspensions, emulsions, syrups, elixirs and pressurized compounds. The active ingredient (e.g., a composition as provided herein) can be dissolved or suspended in a pharmaceutically acceptable liquid carrier such as water, an organic solvent, a mixture of both or pharmaceutically acceptable oils or fats. The liquid carrier can comprise other suitable pharmaceutical additives such as solubilizers, emulsifiers, buffers, preservatives, sweeteners, flavoring agents, suspending agents, thickening agents, colors, viscosity regulators, stabilizers or osmo-regulators.

In alternative embodiments, solid carriers are used to manufacture or formulate compounds as provided herein, or a composition used to practice the methods as provided herein, including solid carriers comprising substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

In alternative embodiments, concentrations of therapeutically active compound in a formulation can be from between about 0.1% to about 100% by weight.

In alternative embodiments, therapeutic formulations are prepared by any method well known in the art, e.g., as described by Brunton et al., eds., Goodman and Gilman's: The Pharmacological Bases of Therapeutics, 12th ed., McGraw-Hill, 2011; Remington: The Science and Practice of Pharmacy, Mack Publishing Co., 20th ed., 2000; Avis et al., eds., Pharmaceutical Dosage Forms: Parenteral Medications, published by Marcel Dekker, Inc., N.Y., 1993; Lieberman et al., eds., Pharmaceutical Dosage Forms: Tablets, published by Marcel Dekker, Inc., N.Y., 1990; and Lieberman et al., eds., Pharmaceutical Dosage Forms: Disperse Systems, published by Marcel Dekker, Inc., N.Y., 1990.

In alternative embodiments, therapeutic formulations are delivered by any effective means appropriated for a particular treatment. For example, depending on the specific antitumor agent to be administered, the suitable means include oral, rectal, vaginal, nasal, pulmonary administration, or parenteral (including subcutaneous, intramuscular, intravenous and intradermal) infusion into the bloodstream. For parenteral administration, antitumor agents as provided herein may be formulated in a variety of ways. Aqueous solutions of the modulators can be encapsulated in polymeric beads, liposomes, nanoparticles or other injectable depot formulations known to those of skill in the art. In alternative embodiments, compounds and compositions used to practice methods as provided herein, are administered encapsulated in liposomes (see below). In alternative embodiments, depending upon solubility, compositions are present both in an aqueous layer and in a lipidic layer, e.g., a liposomic suspension. In alternative embodiments, a hydrophobic layer comprises phospholipids such as lecithin and sphingomyelin, steroids such as cholesterol, more or less ionic surfactants such a diacetylphosphate, stearylamine, or phosphatidic acid, and/or other materials of a hydrophobic nature.

The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, e.g., the latest edition of Remington's Pharmaceutical Sciences, Maack Publishing Co., Easton Pa. (“Remington's”). For example, in alternative embodiments, compounds and compositions used to practice methods as provided herein, are formulated in a buffer, in a saline solution, in a powder, an emulsion, in a vesicle, in a liposome, in a nanoparticle, in a nanolipoparticle and the like. In alternative embodiments, the compositions can be formulated in any way and can be applied in a variety of concentrations and forms depending on the desired in vivo, in vitro or ex vivo conditions, a desired in vivo, in vitro or ex vivo method of administration and the like. Details on techniques for in vivo, in vitro or ex vivo formulations and administrations are well described in the scientific and patent literature. Formulations and/or carriers used to practice embodiments as provided herein can be in forms such as tablets, pills, powders, capsules, liquids, gels, syrups, slurries, suspensions, etc., suitable for in vivo, in vitro or ex vivo applications.

In practicing embodiments as provided herein, the compounds (e.g., formulations) as provided herein can comprise a solution of compositions disposed in or dissolved in a pharmaceutically acceptable carrier, e.g., acceptable vehicles and solvents that can be employed include water and Ringer's solution, an isotonic sodium chloride. In addition, sterile fixed oils can be employed as a solvent or suspending medium. For this purpose any fixed oil can be employed including synthetic mono- or diglycerides, or fatty acids such as oleic acid. In one embodiment, solutions and formulations used to practice embodiments as provided herein are sterile and can be manufactured to be generally free of undesirable matter. In one embodiment, these solutions and formulations are sterilized by conventional, well known sterilization techniques.

The solutions and formulations used to practice methods as provided herein can comprise auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and can be selected primarily based on fluid volumes, viscosities and the like, in accordance with the particular mode of in vivo, in vitro or ex vivo administration selected and the desired results.

The compounds and compositions used to practice methods as provided herein, can be delivered by the use of liposomes. In alternative embodiments, by using liposomes, particularly where the liposome surface carries ligands specific for target cells or organs, or are otherwise preferentially directed to a specific tissue or organ type, one can focus the delivery of the active agent into a target cells in an in vivo, in vitro or ex vivo application.

The compounds and compositions a used to practice methods as provided herein, can be directly administered, e.g., under sterile conditions, to an individual (e.g., a patient) to be treated. The modulators can be administered alone or as the active ingredient of a pharmaceutical composition. Compositions and formulations as provided herein can be combined with or used in association with other therapeutic agents. For example, an individual may be treated concurrently with conventional therapeutic agents.

### Nanoparticles, Nanolipoparticles and Liposomes

Provided are nanoparticles, nanolipoparticles, vesicles and liposomal membranes comprising compounds and compositions used to practice the methods and embodiments as provided herein. Provided are multilayered liposomes comprising compounds used to practice embodiments as provided herein, e.g., as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition used to practice embodiments as provided herein.

Liposomes can be made using any method, e.g., as described in Park, et al., U.S. Pat. Pub. No. 20070042031, including the method of producing a liposome by encapsulating an active agent (e.g., compounds and compositions used to practice methods as provided herein), the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice embodiments as provided herein comprise a substituted ammonium and/or polyanions, e.g., for targeting delivery of a compound used to practice methods as provided herein, to a desired cell type or organ, e.g., brain, as described e.g., in U.S. Pat. Pub. No. 20070110798.

Provided are nanoparticles comprising compounds as provided herein, e.g., used to practice methods as provided herein in the form of active agent-containing nanoparticles (e.g., a secondary nanoparticle), as described, e.g., in U.S. Pat. Pub. No. 20070077286. In one embodiment, provided are nanoparticles comprising a fat-soluble active agent used to practice embodiments as provided herein, or a fat-solubilized water-soluble active agent to act with a bivalent or trivalent metal salt.

In one embodiment, solid lipid suspensions can be used to formulate and to deliver compositions used to practice embodiments as provided herein to mammalian cells in vivo, in vitro or ex vivo, as described, e.g., in U.S. Pat. Pub. No. 20050136121.

### Delivery Vehicles

In alternative embodiments, any delivery vehicle can be used to practice the methods as provided herein, e.g., to deliver compounds and compositions used to practice methods as provided herein, to mammalian cells, e.g., in vivo, in vitro or ex vivo. For example, delivery vehicles comprising polycations, cationic polymers and/or cationic peptides, such as polyethyleneimine derivatives, can be used e.g. as described, e.g., in U.S. Pat. Pub. No. 20060083737.

In one embodiment, a dried polypeptide-surfactant complex is used to formulate compounds and compositions used to practice embodiments as provided herein, e.g. as described, e.g., in U.S. Pat. Pub. No. 20040151766.

In one embodiment, compounds and compositions used to practice methods as provided herein, can be applied to cells using vehicles with cell membrane-permeant peptide conjugates, e.g., as described in U.S. Pat. Nos. 7,306,783; 6,589,503. In one aspect, the composition to be delivered is conjugated to a cell membrane-permeant peptide. In one embodiment, the composition to be delivered and/or the delivery vehicle are conjugated to a transport-mediating peptide, e.g., as described in U.S. Pat. No. 5,846,743, describing transport-mediating peptides that are highly basic and bind to poly-phosphoinositides.

In one embodiment, electro-permeabilization is used as a primary or adjunctive means to deliver the composition to a cell, e.g., using any electroporation system as described e.g. in U.S. Pat. Nos. 7,109,034; 6,261,815; 5,874,268.

### Dosaging

The pharmaceutical compositions and formulations as provided herein can be administered for prophylactic and/or therapeutic treatments. In therapeutic applications, compositions are administered to a subject, e.g., a human in need thereof, in an amount of the agent sufficient to cure, alleviate or partially arrest the clinical manifestations and/or its complications (a “therapeutically effective amount”).

The amount of pharmaceutical composition adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. Dosage levels may range from about 0.01 mg per kilogram to about 100 mg per kilogram of body weight. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods as provided herein are correct and appropriate.

### Products of Manufacture and Kits

Provided are products of manufacture and kits for practicing methods as provided herein, including e.g., a compound 147, or a pharmaceutically acceptable salt or solvate thereof, or optical isomer thereof, or racemic mixture or enantiomer thereof, and optionally also including instructions for practicing methods as provided herein.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1

### Treatments Pharmacologically Activating ATF6 by Administration of Compound 147 Transcriptionally Reprograms Cellular Proteostasis to Mitigate Pathology in a Murine Heart Disease Model

This example demonstrates that methods and compositions as provided herein are effective for a treatment comprising the pharmacologic activation of ATF6 to transcriptionally reprogram cellular proteostasis to mitigate pathology in a heart disease model.

We recently identified a compound that we call “147” (or “compound 147”) in a high-throughput cell-based reporter screen, where it was shown to selectively induce only the ATF6 arm of the unfolded protein response (UPR)¹². Here, we examined the effects of pharmacological activation of ATF6 with compound 147 in a mouse model of acute myocardial infarction (AMI). We found that intravenous administration of compound 147 concurrently with AMI robustly and selectively activated ATF6 and downstream genes of the ATF6 gene program and protected the heart from ischemia/reperfusion (I/R) injury or damage; however, this protection was lost upon the genetic deletion of ATF6. Moreover, compound 147 had no deleterious effects in the absence of pathology, or in other tissues that were unaffected by I/R, an indicator of its safety. Remarkably, we found that by activating ATF6, compound 147 protected other tissues, including the brain, kidney, and liver, when they were subjected to maneuvers that induced I/R damage and impaired proteostasis. This is the first in vivo characterization of any compound that selectively activates a single arm of the UPR, demonstrating that compound 147 provides a novel therapeutic approach for treating I/R damage in a wide range of tissues.

### Results:

ATF6 in Cardiac Myocytes Protects the Heart from I/R Injury:

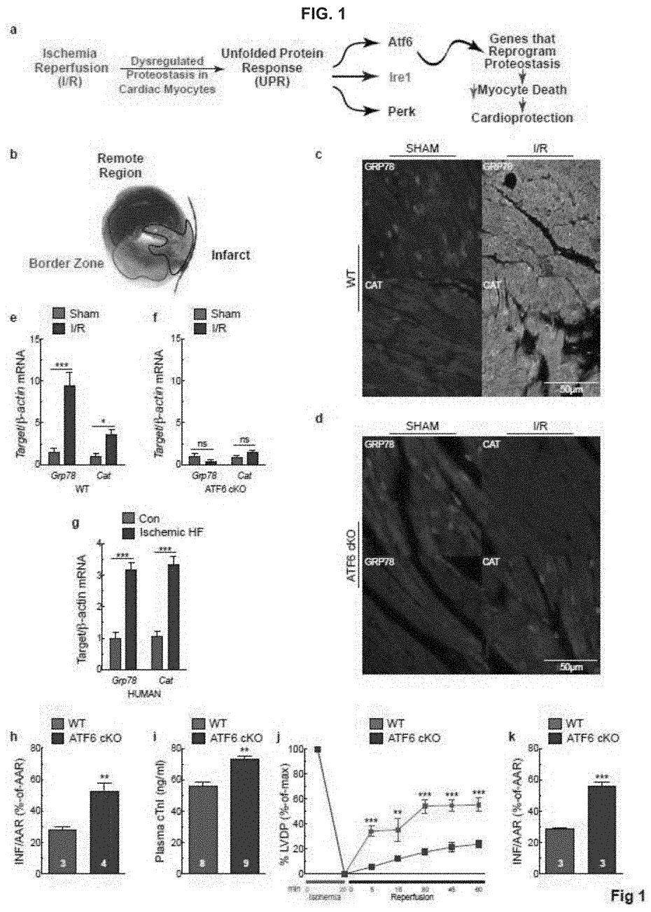

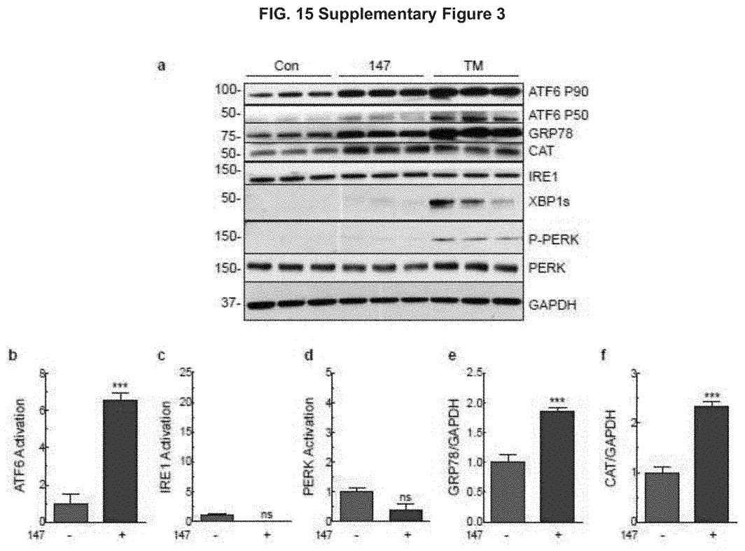

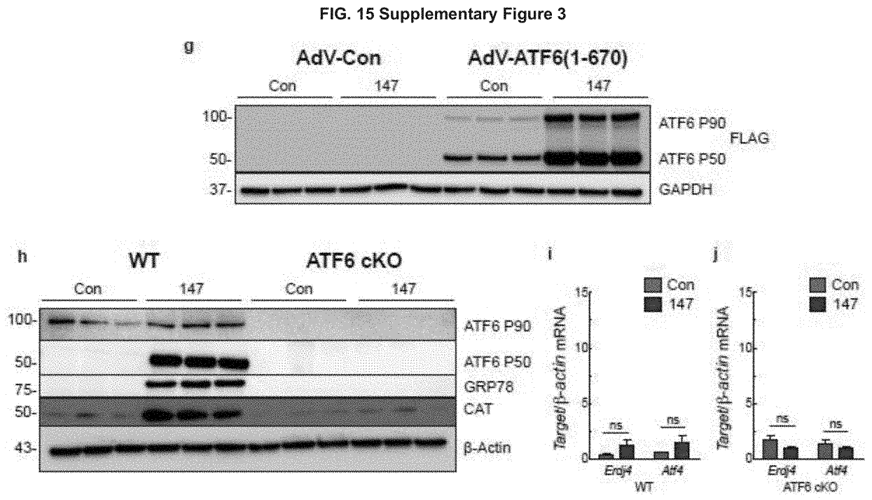

Given their roles in contraction, the viability of cardiac myocytes is crucial for heart function, and it is the loss of cardiac myocytes during ischemia/reperfusion (I/R) that leads to impairment of this function¹⁶. Accordingly, we examined the effects of I/R on proteostasis in isolated cardiac myocytes and in the mouse heart, positing that I/R dysregulates proteostasis, leading to activation of all three arms of the unfolded protein response (UPR), and that the ATF6 arm induces genes that adaptively reprogram proteostasis, decrease myocyte death and provide cardioprotection from I/R damage (FIG. 1A). Consistent with this hypothesis was our finding that I/R activated ATF6, as well as the IRE1 and PERK arms of the UPR in cultured cardiac myocytes (FIG. 13A-C, or supplementary FIG. 1A-D). As a measure of ATF6 activation, we examined expression levels of two known ATF6 target genes, glucose regulated protein 78 kDa (Grp78), a well-studied ER HSP70 chaperone, also known as BiP¹⁹, which participates in ER protein folding, and catalase (Cat), one of the prominent antioxidant genes recently shown to be induced by ATF6^{10,20}. In accordance with the increased activity of ATF6 in response to I/R, both Grp78 and Cat were induced in cultured cardiac myocytes (FIG. 13A, 13E-G, or supplementary FIG. 1a, e, f, g).

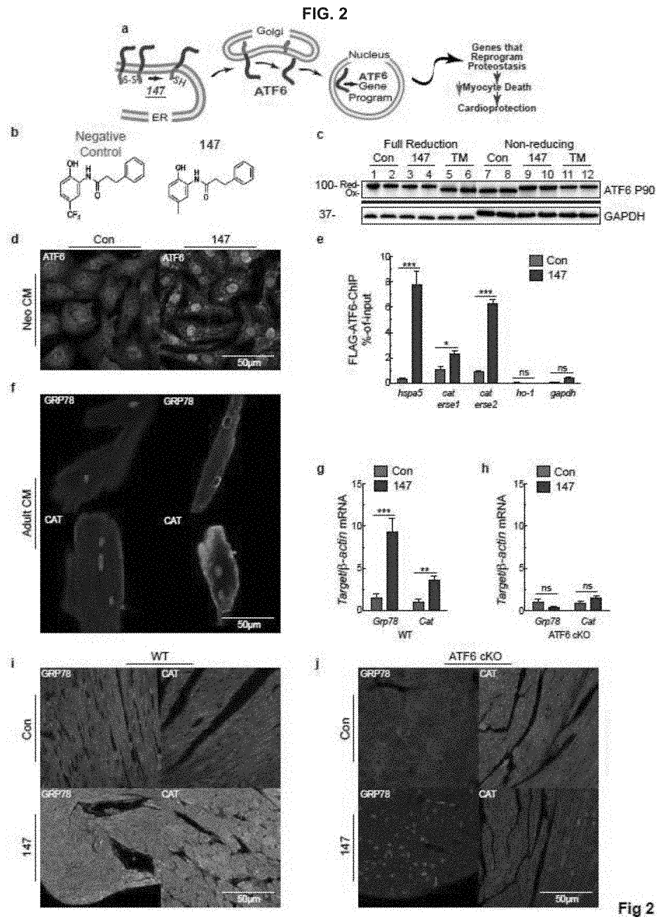

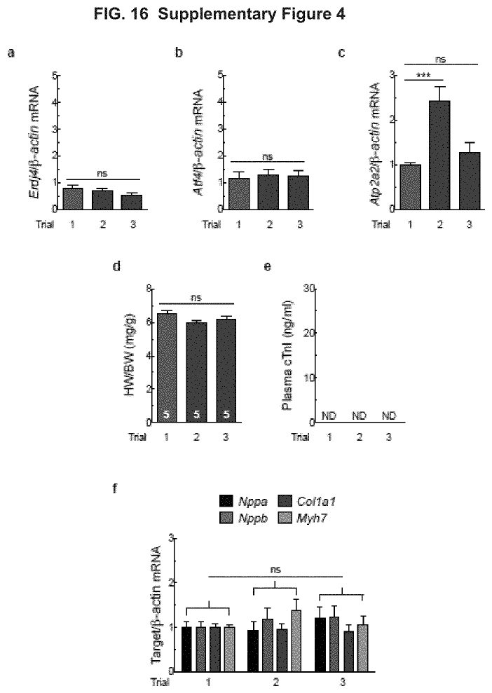

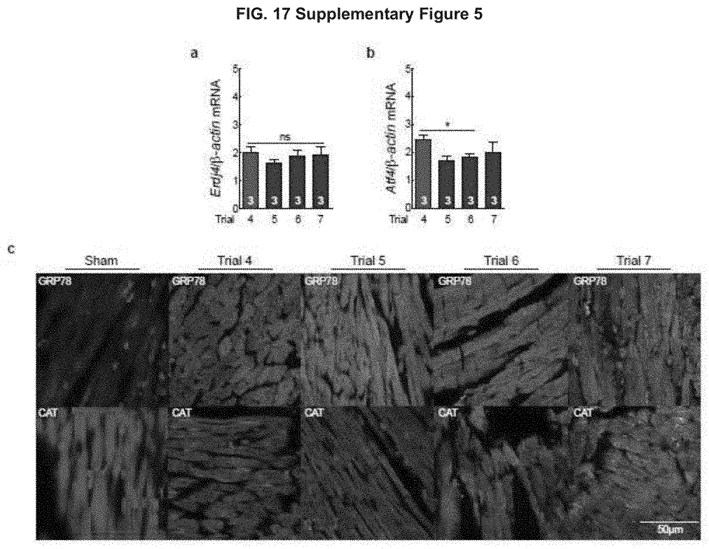

To examine the effects of deleting ATF6 specifically from cardiac myocytes, in vivo, we made an ATF6 conditional knockout mouse (ATF6 cKO) in which Atf6 was selectively deleted in cardiac myocytes of ATF6^{fl/fl} mice using AAV9-cTnT-CRE (FIG. 14A-B, or supplementary FIG. 2a, b). ATF6 cKO and wild type (WT) mice, the latter of which retain ATF6, were subjected to 30 min of surgical coronary artery ligation, followed by 24 hours of reperfusion (i.e. acute I/R), which mimics the reperfusion injury in acute myocardial infarction (AMI) patients that occurs acutely, a time during which the extent of reperfusion injury is progressive²¹. In this model, I/R causes cardiac myocyte death and irreparable damage in the infarct zone (FIG. 1b black), where blood flow has been completely occluded. However, cardiac myocytes adjacent to the infarct, in the border zone (FIG. 1B, outlined in red, or in the lower half of the image), are exposed to sub-lethal I/R and mount protective stress responses, such as the UPR, while the remote region (FIG. 1B, outlined in blue, or in the upper half of the image) is relatively unaffected^{13,22}. Thus, protective stress responses in border zone myocytes conserve their viability, thereby reducing the size of the infarct. In response to AMI, wild type (WT) mice exhibited a robust activation of ATF6, as evidenced by induction of the ATF6 target genes, Grp78 and Cat in the border zone of hearts subjected to acute I/R (FIG. 1C, FIG. 1E); however, this induction was lost in ATF6 cKO mice (FIG. 1D, FIG. 1F). In contrast, the IRE1 target gene, Erdj4, and PERK target gene, Atf4, were similarly induced by I/R in WT and ATF6 cKO mouse hearts (FIG. 14C-D, or supplementary FIG. 2c, d).

However, compared to WT, ATF6 cKO mice had increased infarct sizes and plasma cardiac troponin I (cTnI), canonical indicators of cardiac injury, and exhibited increased ROS-induced damage (FIG. 1h, i; FIG. 14E, or supplementary FIG. 2e). Grp78 and Cat were also increased in hearts from patients with ischemic heart disease (FIG. 1g), supporting the relevance of the ATF6 adaptive arm of the UPR in human pathology and validating the phenotypes observed in our mouse model of AMI. Thus, while all three arms of the UPR were activated in the ischemic mouse heart, cardiac specific deletion of Atf6 significantly increased heart damage in the acute I/R model, demonstrating the importance of the ATF6 arm of the UPR in mitigating I/R injury in this model.

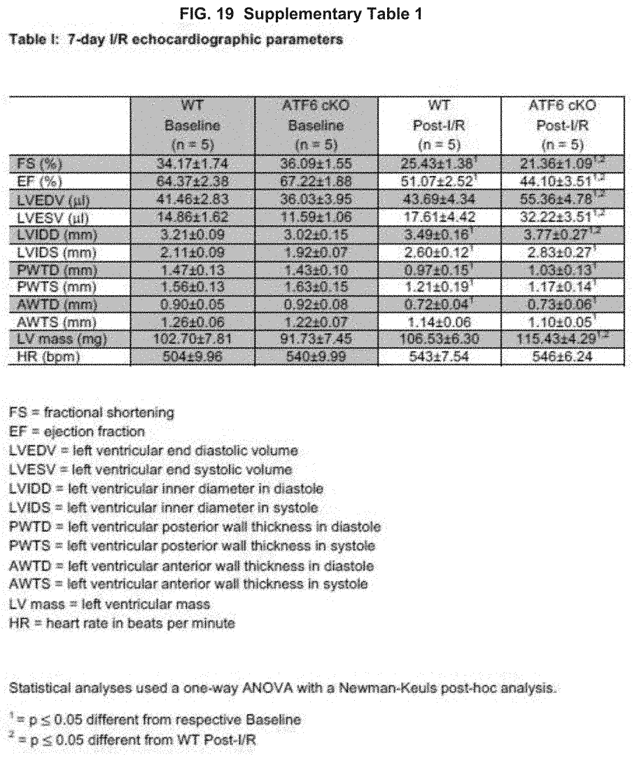

In the days following AMI, the infarct remodels and becomes fibrotic scar tissue, so the detrimental effects of I/R on cardiac function and performance are often more pronounced¹³. Therefore, to examine the effect of Atf6 deletion on cardiac function and performance, mice were analyzed 7 after AMI (i.e. chronic I/R). ATF6 cKO mice exhibited significantly reduced fractional shortening compared to WT, despite being aphenotypic at baseline (FIG. 14F, or supplementary FIG. 2f; FIG. 19, or supplementary Table 1). ATF6 cKO mice also exhibited exaggerated pathological cardiac hypertrophy and plasma cTnI (FIG. 14G-H, or supplementary FIG. 2g-h). Notably, the levels of Grp78 and Cat were lower in ATF6 cKO than WT mice at 7 days (FIG. 14I-J, or supplementary FIG. 2i-j), providing additional evidence of the ATF6 dependence of the induction of adaptive genes in the chronic I/R model.

Cardiac hemodynamics were also assessed in an ex vivo isolated perfused heart model that enables the precise measurement of the strength of cardiac pump function, i.e., left ventricular developed pressure (LVDP), with each contraction in response to I/R injury¹⁰. ATF6 cKO mouse hearts exhibited significantly lower recovery of LVDP and larger infarcts than WT hearts (FIG. 1j, k). Collectively, these results show that ATF6 in cardiac myocytes protects from myocardial I/R injury.

Interestingly, I/R activated ATF6 less than tunicamycin, which is a strong, chemical inducer of ER protein misfolding and UPR activation (FIG. 13A, or supplementary FIG. 1a). Importantly, this result suggests that during I/R there is a reserve of inactive ATF6 remaining that could still be activated. Accordingly, we hypothesize that selective pharmacologic activation of ATF6 could supplement the modest ATF6 activation achieved by I/R to enhance cardioprotection.

Compound 147 Activates ATF6 and Induces ATF6-Target Genes in Cardiac Myocytes:

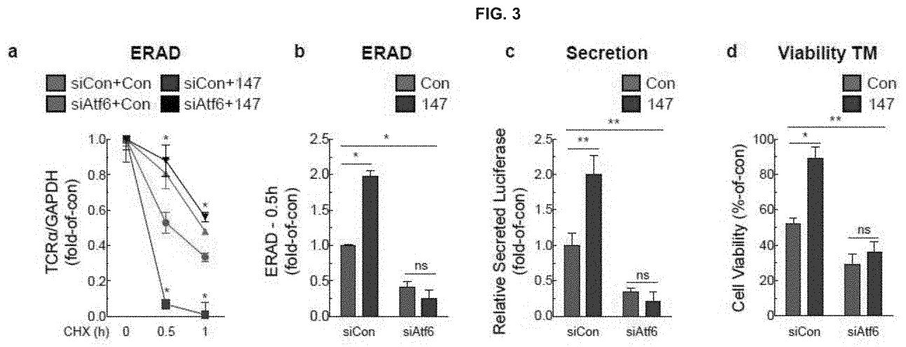

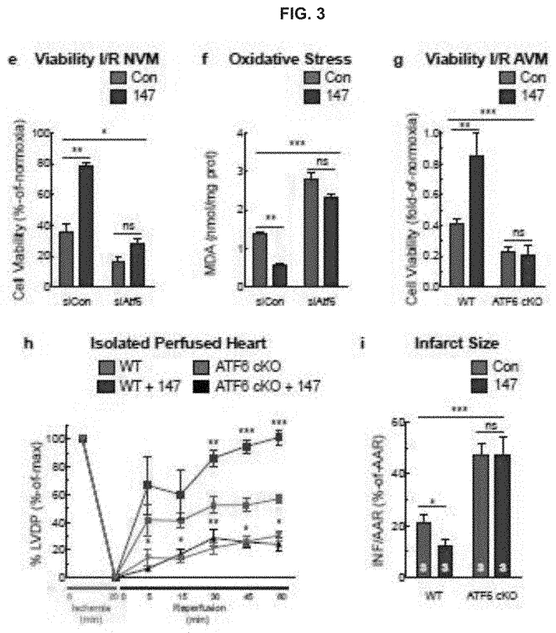

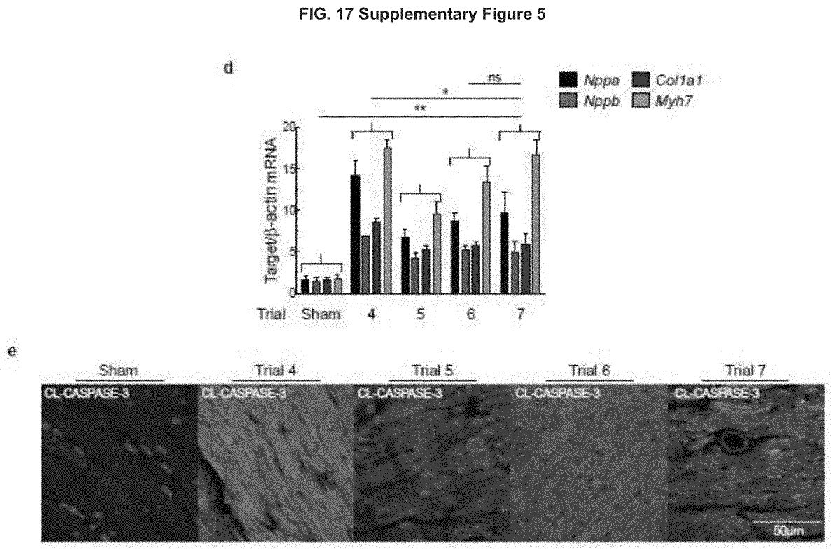

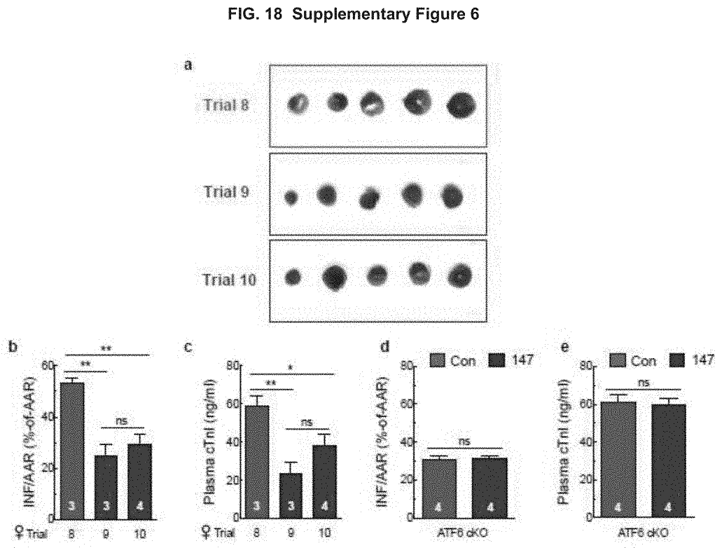

The compound 147 was previously shown to specifically activate ATF6 in HEK293 cells through a canonical mechanism involving translocation of ATF6 from the ER to the Golgi, where it is cleaved by S1 and S2 proteases to release the active ATF6 transcription factor²³ (FIG. 2a). The translocation of ATF6 out of the ER during protein misfolding is known to require a reduction of the inter- and intramolecular disulfide bonds in ATF6; however, neither the effects of compound 147 on ATF6, nor its mechanism of action have been studied in cardiac myocytes. Here, in cultured cardiac myocytes, a control compound that closely resembles compound 147 (FIG. 2b), but does not activate ATF6, did not affect the disulfide bond status of ATF6, while compound 147 reduced intramolecular disulfide bonds in ATF6 (FIG. 2c, lanes 7-10). Moreover, while the control compound did not activate any of the UPR pathways, exemplary compound 147 activated ATF6, but not PERK or IRE1 (FIG. 15A-D, or supplementary FIG. 3A-D). Thus, in cardiac myocytes, compound 147 induced the canonical reduction of disulfide bonds in ATF6, which is associated with ATF6 translocation to the Golgi. Coordinate with the generation of the active, nuclear form of ATF6 in the Golgi was our finding that compound 147 increased the nuclear translocation of ATF6 in cardiac myocytes (FIG. 2d), increased the cleavage, activation, and association of ATF6 with known ATF6 binding sites in the Grp78 and Cat promoters (FIG. 2e; FIG. 15G, or supplementary FIG. 3g), and increased mRNA levels of both genes (FIG. 2f; FIG. 15B, FIG. 15E-F, or supplementary FIG. 3B, FIG. 3E-F). Intravenous administration of compound 147 activated ATF6 and increased Grp78 and Cat expression in WT mouse hearts; however, this effect was completely absent in ATF6 cKO mice (FIG. 2G-J; FIG. 15H, or supplementary FIG. 3H). As a testament to the ability of exemplary compound 147 to activate only the ATF6 arm of the UPR was our finding that 147 had no effect on the expression levels of the IRE1 or PERK targets, Erdj4 or Atf4 in either WT or ATF6 cKO mouse hearts (FIG. 15I-J, or supplementary FIG. 3I-J). Thus, 147 selectively activates the ATF6 arm of the UPR in the heart, in vivo, as it does in cultured cardiac myocytes.

Compound 147 Improves ER Proteostasis and Decreases Oxidative Stress:

Mechanistically, we examined whether exemplary compound 147 could replicate the breadth of adaptive effects of ATF6 on ER proteostasis, such as increasing ER associated protein degradation (ERAD), which removes potentially toxic terminally misfolded proteins, increasing folding and consequent secretion of proteins made in the ER, and enhancing protection against ER protein misfolding. Here, compound 147 increased ERAD, as measured by the rate of degradation of ectopically expressed TCRα (FIG. 3a, b), increased secretion of a protein folded in the ER as is transported through the conventional secretory pathway, as determined by secretion of ectopically expressed Gaussia luciferase (FIG. 3c), and protected cells from death in response to ER protein misfolding induced by tunicamycin (FIG. 3d); importantly, all of these effects were lost upon knockdown of Atf6. Next, we explored whether exemplary compound 147 could replicate the adaptive effects of ATF6 against oxidative stress, in vitro. Exemplary compound 147 significantly improved survival of cardiac myocytes subjected to I/R (FIG. 3e) and decreased lipid peroxidation (FIG. 3f), a measure of ROS-mediated damage; importantly, these effects of compound 147 were, again, lost upon knockdown of Atf6. Thus, exemplary compound 147 replicated a broad spectrum of the adaptive effects of ATF6 on proteostasis and oxidative stress; moreover, all of these effects required endogenous ATF6, demonstrating the ATF6-dependent mechanism of action of compound 147.

Compound 147 Administered In Vivo Protects Isolated Cardiac Myocytes and Perfused Hearts:

In an initial experiment to determine whether exemplary compound 147 retained its ability to protect myocytes in vivo, mice were treated for 24 h with either the negative control compound or exemplary compound 147, after which cardiac myocytes were isolated and subjected to I/R in culture. Compared to the negative control, myocytes from 147-treated WT mice exhibited increased viability when subjected to I/R in vitro (FIG. 3g, left); however, this benefit was absent in myocytes prepared from ATF6 cKO mice (FIG. 3g, right). This demonstrated that when administered in vivo, exemplary compound 147 retained its ability to protect cardiac myocytes from I/R damage in culture, and this protection was mediated through endogenous ATF6. To determine whether the protection seen in isolated cardiac myocytes had any effect in the intact heart, hearts from WT and ATF6 cKO mice that had been treated for 24 h with compound 147 were examined in the ex vivo I/R model. Compared to control, hearts from compound 147-treated WT mice had greater LVDP recovery and smaller infarct sizes (FIG. 3h, blue vs red; 3i, left). Notably, compound 147 exhibited neither of these beneficial effects in hearts from ATF6 cKO mice (FIG. 3h, gray and black; 3i, right). Thus, when administered to mice, compound 147 protected cardiac myocytes, and decreased I/R injury of the heart, while preserving cardiac function. Furthermore, all of these beneficial effects of exemplary compound 147 were dependent upon endogenous ATF6 in cardiac myocytes.

Compound 147 Induces ATF6 Target Genes in the Heart:

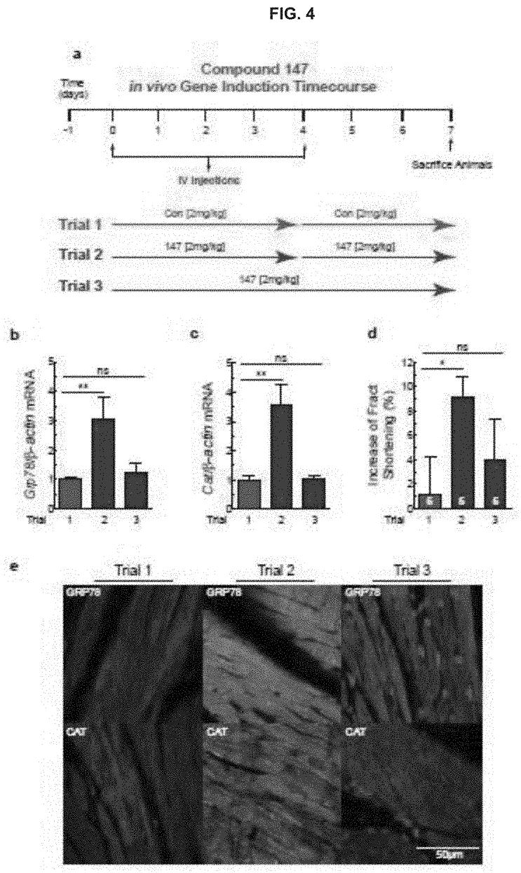

Next, the effects of compound 147 on ATF6 target gene induction in the hearts of mice that were not subjected to I/R were examined using several dosing protocols over the span of 7 days (FIG. 4a). Mice were injected with the negative control compound or compound 147 either twice, at days 0 and 4 (Trials 1 and 2, respectively), or compound 147 was injected only once, at day 0 (Trial 3). Compared to Trial 1, Trial 2 resulted in increased the expression of the ATF6-regulated genes Grp78 and Cat, but not the IRE1-regulated Erdj4 or the PERK-regulated Atf4 (FIG. 4b, c, e; FIG. 16A-B, or supplementary FIG. 4a, b; Trial 1 vs 2). No significant gene induction was seen upon Trial 3 (FIG. 16A-B, or supplementary FIG. 4a, b; Trial 1 vs 3), indicating that compound 147-mediated induction of ATF6-target genes is transient.

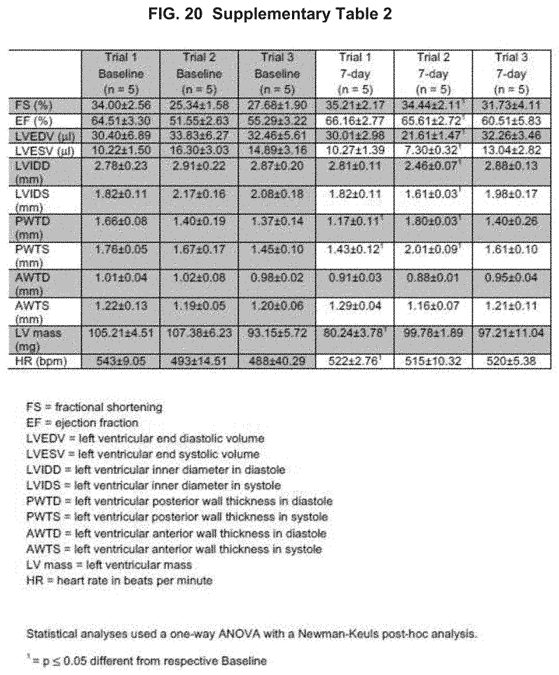

Interestingly, Trial 2 significantly enhanced cardiac performance (FIG. 4d; Trial 1 vs 2; FIG. 20, or supplementary Table 2), which could be partly due to compound 147-dependent increases in Atp2a2 expression (FIG. 16C, or supplementary FIG. 4c). Atp2a2 encodes SERCA2a, an adaptive SR/ER-localized calcium ATPase previously shown to be ATF6-inducible in the heart²⁴ and to improve contractility in heart failure patients²⁵. None of the dosing protocols resulted in pathological cardiac hypertrophy, increased plasma cTnI or expression of pathology-associated genes, such as Nppa, Nppb, Col1a1 or Myh7 (FIG. 16D-F, or supplementary FIG. 4d-f). This indicates that 147 does not induce cardiotoxicity over the course of 7 days. Furthermore, no apparent deficits were observed in any of the trials upon inspection of the liver or kidneys when steatosis or glomerular filtration rate was assayed by hepatic triglyceride accumulation or creatinine clearance (data not shown). Thus, the effects of 147 on ATF6-target gene induction were transient, lasting ˜3 days. Moreover, 147 had no untoward effects on other organ systems, and even in the absence of a pathological maneuver, compound 147 had beneficial effects on heart function.

Compound 147 Protects the Heart from Chronic I/R Injury In Vivo:

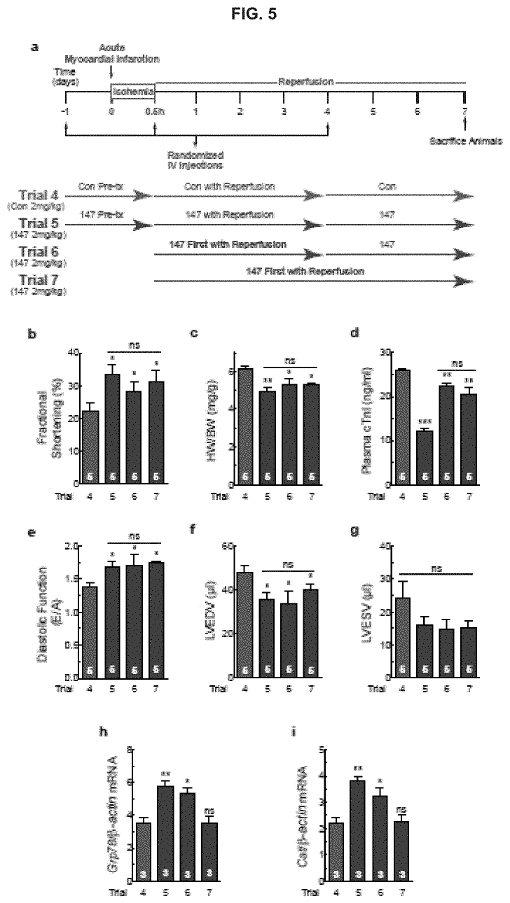

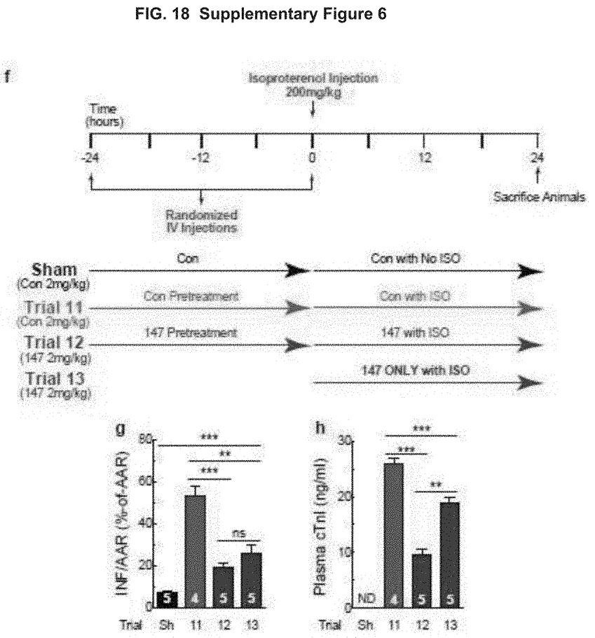

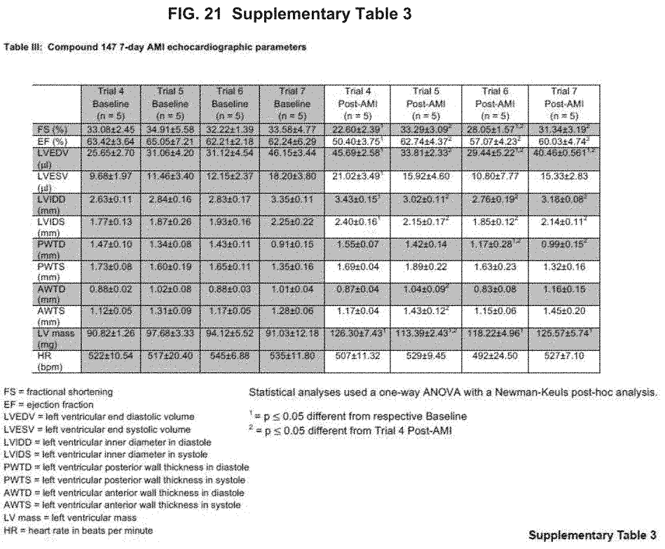

Next, the effects of compound 147 were examined in the chronic I/R model (FIG. 5a). In Trials 4 and 5, the negative control compound or compound 147 were administered 24 h prior to AMI, with a second dose at reperfusion and a third dose 4 days later. In Trial 6, compound 147 was administered at reperfusion and again 4 days later. In Trial 7, compound 147 was administered only one time at reperfusion. Trials 6 and 7 were specifically designed and implemented to mimic the timing of the treatment of choice for AMI patients. Given the transient nature of compound 147, we designed our multiple-dose strategy so that it mimics a therapeutic approach used for treating AMI patients as soon as possible after the infarction, to mitigate the initial reperfusion damage to the heart, as well as days later, to ameliorate the detrimental effects of cardiac remodeling in the infarct and infarct border zones on heart pump function. Strikingly, cardiac performance was preserved to similar extents in all trials of compound 147, as was the ability of compound 147 to reduce pathological cardiac hypertrophy (FIG. 5b, c). Compound 147 decreased plasma cTnI in all trials, though somewhat less so in Trials 6 and 7 (FIG. 5d). Importantly, exemplary compound 147 preserved diastolic cardiac function and left ventricular dilatation in all of the trials (FIG. 5e-g; FIG. 21, or supplementary Table 3), showing that exemplary compound 147 reduces the progression toward heart failure. In Trials 5 and 6 the beneficial structural and functional effects were accompanied by increased expression of the ATF6-regulated genes, Grp78 and Cat, but not Erdj4 and Atf4 (FIG. 5h, i; FIG. 17A-C, or supplementary FIG. 5a-c); however, in Trial 7, expression of Grp78 and Cat were comparable to control treated animals, as expected given the transient nature of exemplary compound 147-mediated gene induction (FIG. 3). Moreover, I/R induced cardiac pathology genes (FIG. 17D, or supplementary FIG. 5d, Sham vs Trial 4); however, these effects were blunted by 147 (FIG. 17D, or supplementary FIG. 5d, Trials 5-7). In addition, decreased levels of pro-apoptotic cleaved caspase-3 were seen in Trials 5-7 (FIG. 17E, or supplementary FIG. 5e), indicating that exemplary compound 147 protected against I/R-induced myocyte apoptosis. Thus, pharmacologic ATF6 activation at reperfusion ameliorated pathologic cardiac dysfunction in response to chronic I/R injury.

Compound 147 is Beneficial in a Wide Range of Proteostasis-Mediated Disease Models, In Vivo:

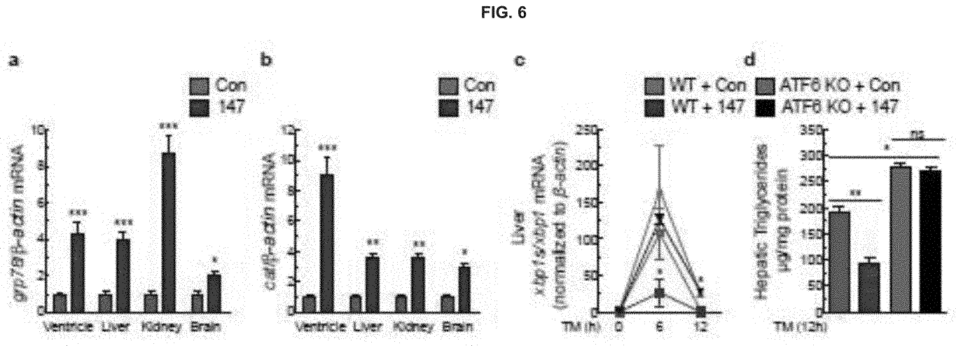

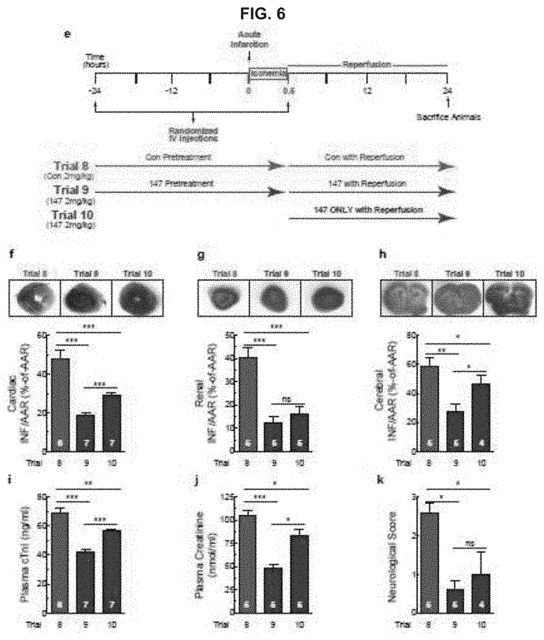

The results in FIG. 5 indicated that exemplary compound 147 had beneficial effects on cardiac function sooner than 7 d after exemplary compound 147 administration. Thus, we examined the effects of compound 147 acutely, 24 h after administration, an important time at which AMI patients are often treated by coronary angioplasty. Additionally, since ATF6 is expressed in all cells, we thought that it might be effective in tissues in addition to the heart. Accordingly, we determined the effects of compound 147 in the heart, as well as other tissues. Within 24 h of exemplary compound 147 administration we found robust activation of ATF6 target genes in the heart, liver, kidney and brain, as evidenced by significant increases in of Grp78 and Cat (FIG. 6a, b), although the magnitude of the responses varied somewhat between these tissues. The functionality of 147-mediated activation of ATF6 in the liver was evident in that it significantly reduced ER protein misfolding, measured by XBP1 splicing, in mice that had been injected with tunicamycin; this beneficial effect was lost upon genetic deletion of ATF6 (FIG. 6c). Further, functionality of 147 in the liver was evident in its ability to reduce hepatic triglycerides, a hallmark of hepatic steatosis, which demonstrates improved ER proteostasis in the liver (FIG. 6d, blue); this beneficial effect of 147 was also lost upon deletion of ATF6 (FIG. 6d, black).

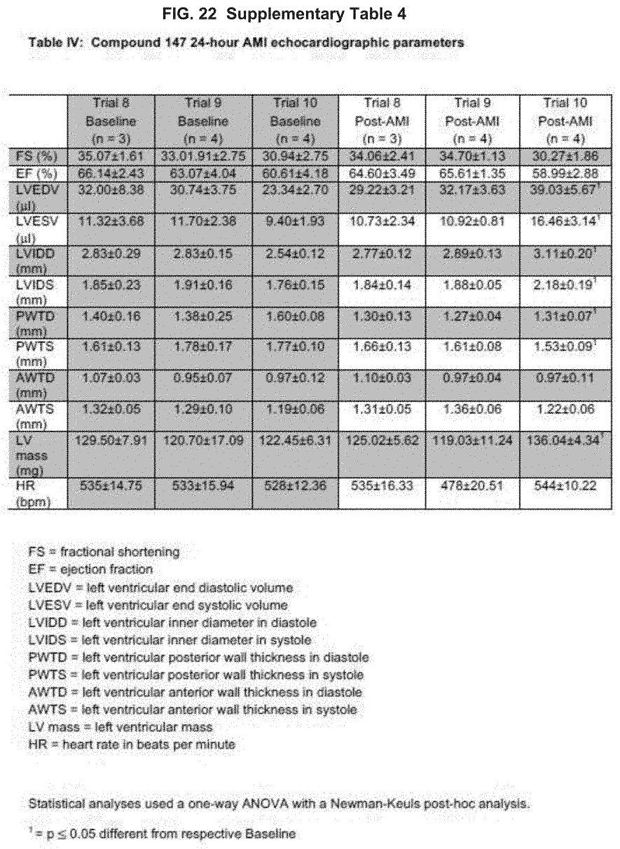

Next, to examine the functional effects of 147 in various tissues, the control compound or 147 were administered, as shown in FIG. 6e, and the effects were examined on tissue damage in the heart via the acute I/R model, the kidney via transient unilateral renal portal system occlusion, and in the brain via transient unilateral middle cerebral artery occlusion. Throughout the studies, the surgeon and the data analyst were blinded to the animal assignments, which were predetermined by a separate investigator. Remarkably, even when administered only at the time of reperfusion, compound 147 significantly decreased infarct sizes in all three tissues (FIG. 6f-j; FIG. 18A, or supplementary FIG. 6a). Moreover, compound 147 decreased plasma cTnI and plasma creatinine, which are biomarkers of cardiac and kidney damage, respectively, and it improved behavioral indicators of post-ischemic neurological deficit (FIG. 6i-k). As expected, since the trial parameters of the acute myocardial I/R protocol are too short for structural remodeling and, thus, an observable function deficit, there was no effect on cardiac performance, chamber size, or pathological hypertrophy as monitored by echocardiography (FIG. 22, or supplementary Table 4). As further proof of concept, the myocardial acute I/R experiment was replicated in female mice and, again, both Trials 9 and 10 conferred protection as evidenced by reduced infarct sizes and plasma cTnI (FIG. 18B-C, or supplementary FIG. 6b, c). Importantly, these beneficial effects of compound 147 in response to myocardial acute I/R were not seen in ATF6 cKO mice, further emphasizing that compound 147-mediated protection of the heart required ATF6 activation (FIG. 18D-E, or supplementary FIG. 6d, e). Interestingly, the beneficial effects of compound 147 were also seen in a different AMI model induced by acute administration of the β-adrenergic receptor agonist, isoproterenol, which is known to cause widespread oxidative damage and cardiac myocyte death in mice at this dose (Supplementary FIG. 6f-h).

Thus, when administered at the time of injury, 147 protected wide range of tissues from I/R damage, emphasizing the broad spectrum of potential applications for this compound as a transcriptional regulator of the ATF6 arm of the UPR and subsequent reprogramming of proteostasis, in vivo.

### Discussion:

Ideally, an effective therapy for AMI should function in a temporally extended manner, acting acutely, to minimize reperfusion damage, and chronically, to influence post-AMI remodeling so as to preserve contractility and prevent heart failure¹⁵. While a number of potential therapies that act acutely to minimize reperfusion damage have been tested, many of them have failed to move through the drug development process and there is still no clinically available intervention¹⁵. We posited that this might be because most of the previous therapeutics function only upon acute I/R. Furthermore, many of the initial trials performed in small animals have not tested therapies at times that accurately mimic typical clinical interventions (i.e. during coronary angioplasty) and have not adhered to the FDA's Good Laboratory Practices (GLP). Accordingly, in addition to addressing these points in the design of our animal trials here, we examined the therapeutic function at both acute and chronic times after I/R. We also set out to develop a therapeutic approach that would exert beneficial effects through multiple mechanisms in various cellular locations, which we felt would broaden the potential utility to include different tissues and widen the scope to multiple proteostasis-based pathologies.

In this regard, we focused on ATF6, since it adaptively reprograms ER proteostasis by inducing a wide range of protective response genes that encode proteins that reside in various cellular locations where some act acutely and others act chronically. Using this strategy, we found that selective pharmacologic activation of only the ATF6 arm of the UPR with compound 147 in mice acted acutely to reduce reperfusion damage in the heart and acted chronically to preserve cardiac function. In addition to demonstrating its efficacy in the ischemic heart, we found that compound 147 protected the liver in a mouse model of dysregulated hepatic proteostasis, and it protected the kidneys and brain in models of renal and cerebral I/R damage. These findings, together with a recent report showing that compound 147 enhances the differentiation of human embryonic stem cells²⁶, support the broad therapeutic potential of pharmacologic activation of ATF6 for treating a wide range of proteostasis-based pathologies in various tissues.

In terms of its suitability as a pharmacologic agent, compound 147 exhibits many desirable properties. For example, compound 147 is highly specific, serving as the first example of a compound that selectively activates only one arm of the UPR, ATF6, which is well known for exerting mainly beneficial effects in many different cell types. Compound 147 is highly efficacious in vivo, functioning at a dose similar to many other cardiovascular drugs and has the capacity to cross the blood brain barrier. Moreover, exemplary compound 147 does not exhibit any apparent toxicity or deleterious off-target effects in vivo. Both the efficacy and tolerance of compound 147 can be attributed in large part to the high-stringency, cell-based transcriptional profiling that was done in the initial screening to ensure that compound 147 specifically activates only the ATF6 arm of the UPR, instead of global UPR activation²³. The relatively transient activation of ATF6 by compound 147 in vivo is also potentially advantageous, since many stress-signaling pathways, including the UPR, can be beneficial initially, but damaging upon chronic activation²⁷. Since I/R only partially activates ATF6, the remaining inactive ATF6 provides a therapeutic reserve for compound 147 to activate, allowing it to boost adaptive ATF6 signaling pathways in multiple tissues, in vivo. Remarkably, we found that 147 exerted beneficial effects in the hearts of mice that were not subjected to any injury maneuvers, underscoring the safety, and perhaps even benefits of the compound in healthy tissues. Thus, while future pharmacokinetic and toxicology studies will address further details of exemplary compound 147 action, it is clear from the results presented here that exemplary compound 147 is easily administered, well tolerated, acts quickly, boosts an endogenous adaptive transcriptional stress signaling pathway, and has no apparent off-target or untoward effects, all of which are attributes of an excellent candidate for therapeutic development.

Impaired proteostasis contributes to numerous pathologies and even impacts aging²⁸. Thus, global improvement of proteome quality through pharmacologic activation of defined transcriptional regulators of proteostasis should ameliorate a broad range of proteostasis-based diseases. Recent findings showing that the sphere of influence of the UPR, in particular, the ATF6 arm of the UPR, extends well beyond the ER to reprogram proteostasis in many cellular locations¹⁰, support the potential broad spectrum of impact of pharmacologic compounds, like exemplary compound 147. The results presented here provide proof-of-principle that this type of pharmacologic correction can be achieved with well-characterized compounds, such as compound 147 that selectively activate a specific protective aspect of UPR signaling.

Figure Legends

FIG. 1: ATF6 in Cardiac Myocytes Protects the Heart from I/R Injury.

FIG. 1A, Activation of the unfolded protein response (UPR) by ischemia/reperfusion (I/R) in the heart. FIG. 1B, Post-AMI cross section of the left ventricle of a mouse heart. FIG. 1C-D: Immunohistochemical (IHC) staining of GRP78 or CAT (cyan), tropomyosin (red), and nuclei (TOPRO-3) in the border zone of wild-type (WT) (FIG. 1C) or ATF6 cKO (FIG. 1D) hearts subjected to either sham or acute I/R surgery. e-g, Quantitative real-time PCR (qPCR) for Grp78 or Cat in sham or border zone of post-I/R hearts in WT (e), ATF6 cKO (FIG. 1F), or in ventricular explants from control or ischemic heart failure patients (FIG. 1G). h,i, Infarct sizes (FIG. 1H) and plasma cardiac troponin I (cTnI) (FIG. 1I) in WT and ATF6 cKO mice post-I/R. j,k, Left ventricular developed pressure (LVDP) (FIG. 1J) and relative infarct sizes (k) post-ex vivo I/R. Data are represented as mean±s.e.m. *P<0.05, **P<0.01, ***P<0.001.

FIG. 2: Compound 147 Selectively Activates ATF6 in the Heart.

a, Diagram of hypothetical mechanism of ATF6 activation by compound 147. b, Chemical structure of synthetic control compound and compound 147. c, Immunoblot of ATF6 and GAPDH in NRVM 24-hours after treatment with compound 147 or TM in fully-reducing condition (lanes 1-6) or non-reducing conditions (lanes 7-12). Shift exhibited in Atf6 in TM-treated cells in full-reducing conditions is typical of de-glycosylated ATF6. d, Immunocytofluorescence (ICF) of ATF6 (green), alpha-actinin (red) and nuclei (TOPRO-3) in NRVM 24-hours after treatment with compound 147. e, Chromatin immunoprecipitation (ChIP-qPCR) of known ATF6 target promoter binding elements (ERSE) for Grp78 (hspa5), cat, and negative control targets Heme oxygenase 1 (ho-1) and gapdh NRVM infected with AdV encoding Flag-ATF6 (1-670) 24-hours after treatment with compound 147. f, ICF of GRP78 and CAT (green), alpha-actinin (red) and nuclei (TOPRO-3) in AMVM 24-hours after treatment with compound 147. g, h, qPCR for Grp78 or Cat in LV of WT (g) or ATF6 cKO (h) hearts 24-hours post-treatment with control or compound 147. i,j, IHC staining of GRP78 or CAT (cyan), tropomyosin (red), and nuclei (TOPRO-3) in left ventricle (LV) of WT (i) or ATF6 cKO (j) hearts 24-hours post-treatment with control or compound 147. Data are represented as mean±s.e.m. *P<0.05, **P<0.01, ***P<0.001.

FIG. 3: Exemplary Compound 147 Improves Proteostasis and Decreases Oxidative Stress in an ATF6-Dependent Manner.

a, b, NRVM were infected with AdV-HA-T-cell antigen receptor alpha-chain (TCR; an ER-transmembrane protein that is chronically misfolded and degraded by ERAD), treated with siCon or siAtf6 and either control or compound 147 for 24-hours prior to cyclohexamide for 0, 0.5 or 1 h. Densitometry of the HA-TCRE immunoblots at the respective times (a) and ERAD at the 0.5-hour time point (b) are shown. c, Secretory proteostasis assayed in NRVM when transfected with Gaussia luciferase and treated with siCon or siAtf6, and either control or compound 147 for 24-hours. Medium was collected and luciferase activity was measured. d, NRVM were transfected with siCon or siAtf6, then treated with or without TM, control or compound 147 for 24 h, after which viability was determined. e, f, NRVM were transfected with siCon or siAtf6, treated with or without control or compound 147 for 24 h, then I/R, after which viability (e) and MDA (f) were measured. g, Viability of I/R-treated cultured adult cardiomyocytes isolated from WT or ATF6 cKO mice 24-hours post-treatment with control or compound 147. h.i, LVDP (h) and relative infarct sizes (i) of WT or ATF6 cKO mice treated 24 h with control or compound 147 then ex vivo I/R. Data are represented as mean±s.e.m. **P<0.01, ***P<0.001.

FIG. 4: Exemplary Compound 147 Gene Induction Timecourse, In Vivo.

a, Experimental design testing the effects of compound 147 in WT untreated mice. b, c, qPCR for Grp78 (b) or Cat (c) in LV of mice from indicated trials. d, Percent increase in fractional shortening. Detailed analyses of echocardiography parameters are in Extended Data Table 2. e, IHC staining of GRP78 or CAT (cyan), tropomyosin (red), and nuclei (TOPRO-3) in LV of mice from respective trials. Data are represented as mean±s.e.m. *P<0.05, **P<0.01.

FIG. 5: Exemplary Compound 147 Improves Cardiac Performance 7 d Post-AMI.

a, Experimental design and dosing protocols for animal trials during remodeling phase of AMI. b, f, g, Echocardiographic parameters of fractional shortening (b), LV end diastolic volume (LVEDV) (f) and LV end systolic volume (LVESV) (g). Detailed analyses of echocardiography parameters are in Extended Data Table 3. c, Ratio of heart weight to body weight. d, Plasma cTnI. e, Diastolic function as determined by pulse wave Doppler (PW) technique to analyze E and A waves. h, i, qPCR for Grp78 (h) or Cat (i) in LV of mice from indicated trials at culmination of study. Data are represented as mean±s.e.m. *P<0.05, **P<0.01, ***P<0.001.

FIG. 6: Exemplary Compound 147 Exerts Widespread Protection in Multiple Organ Systems.

a, b, qPCR for Grp78 (a) or Cat (b) in left ventricular, liver, kidney, and brain extracts from WT mice 24-hours post-treatment with control or compound 147. c, Ratio of transcript levels of Xbp1s to Xbp1 as determined by qPCR in liver extracts from WT or ATF6 KO mice 24-hours post-treatment with control or compound 147 and then treated with 2 mg/kg of TM for designated periods of time. d, Triglyceride levels in liver extracts from WT or ATF6 KO mice 24-hours post-treatment with control or compound 147 and then treated with 2 mg/kg of TM for 12-hours. e, Preclinical experimental design testing protective effects of compound 147. f-h, Relative infarct sizes in the heart (f), kidney (g), and brain (h) of male mice 24 h after reperfusion. i-k, Plasma cTnI (i), plasma creatinine (j), and neurological score (k) based on the Bederson system of behavioral patterns post-cerebral ischemic injury of male mice 24 h after reperfusion of respective injury models. Data are represented as mean±s.e.m. **P<0.01, ***P<0.001.

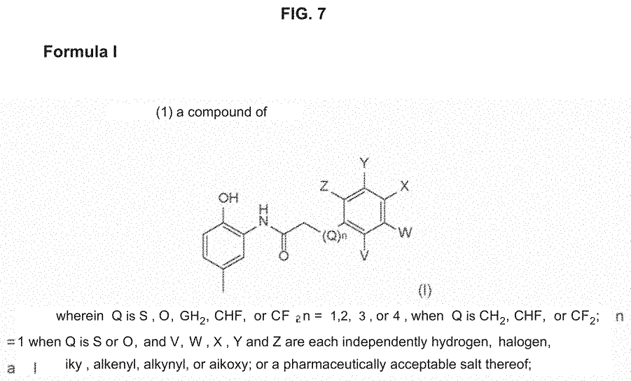

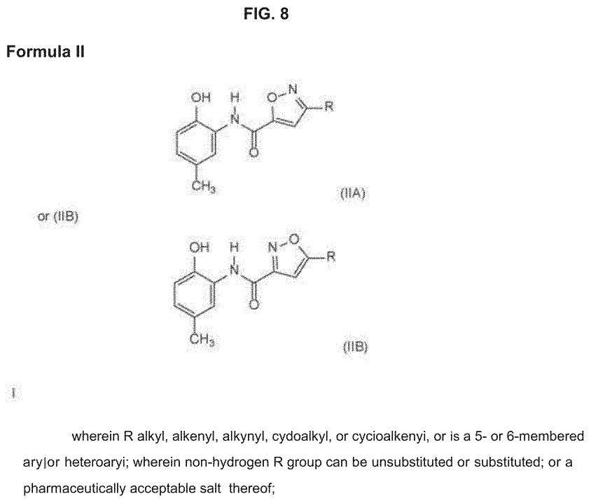

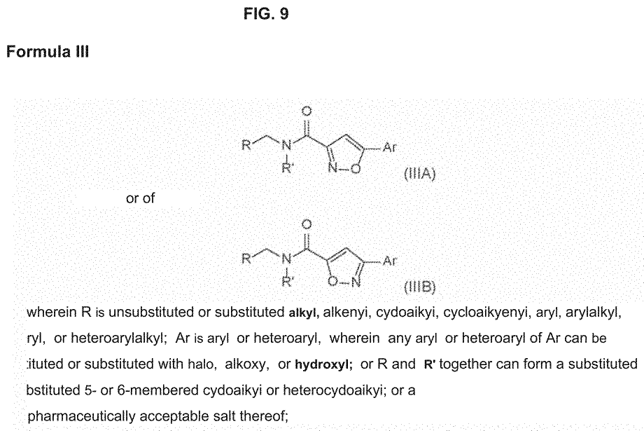

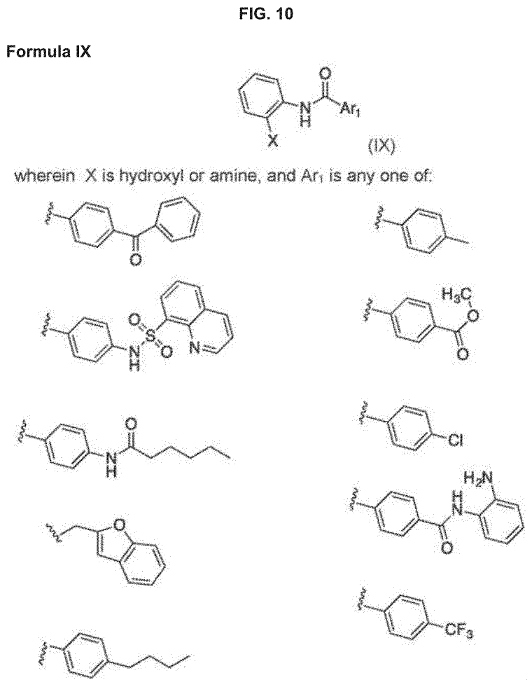

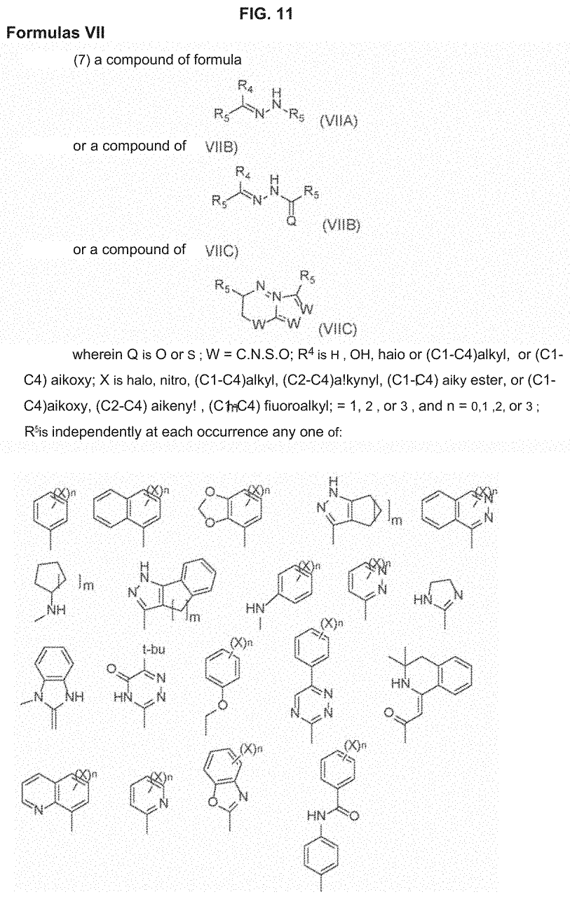

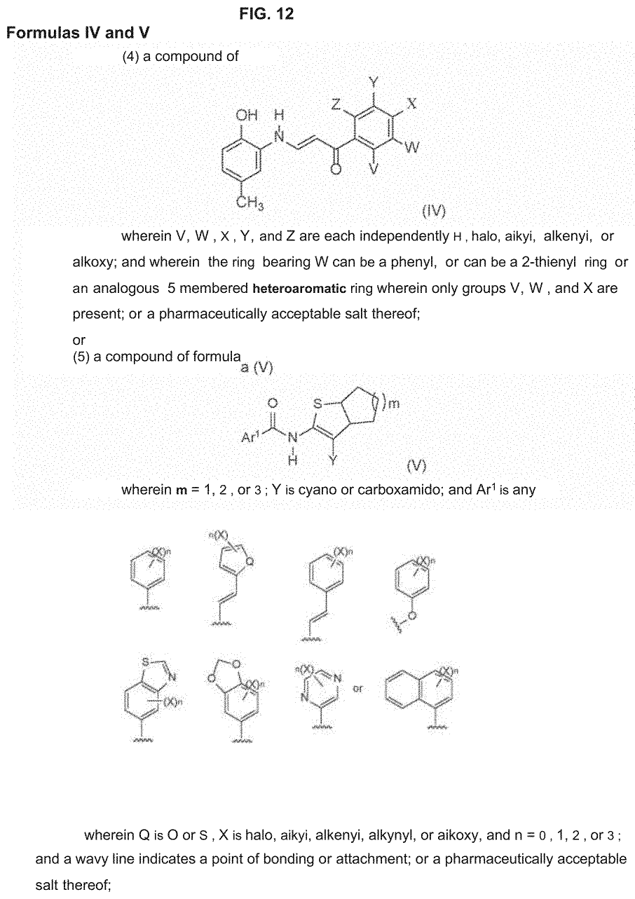

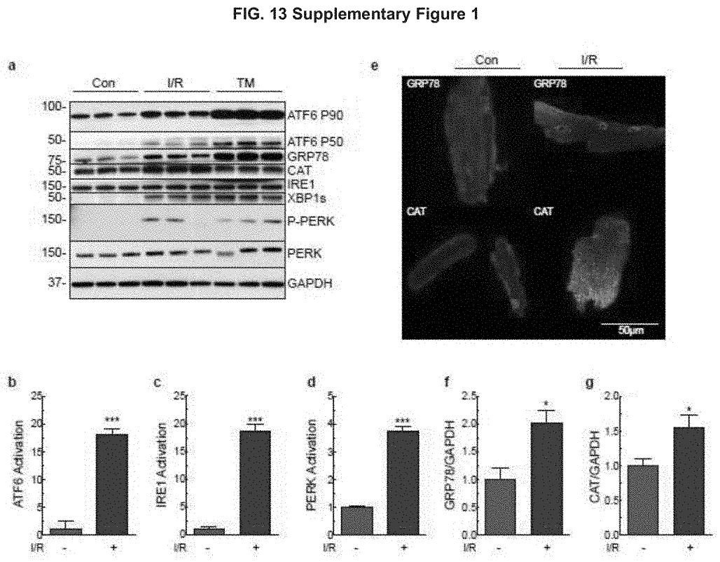

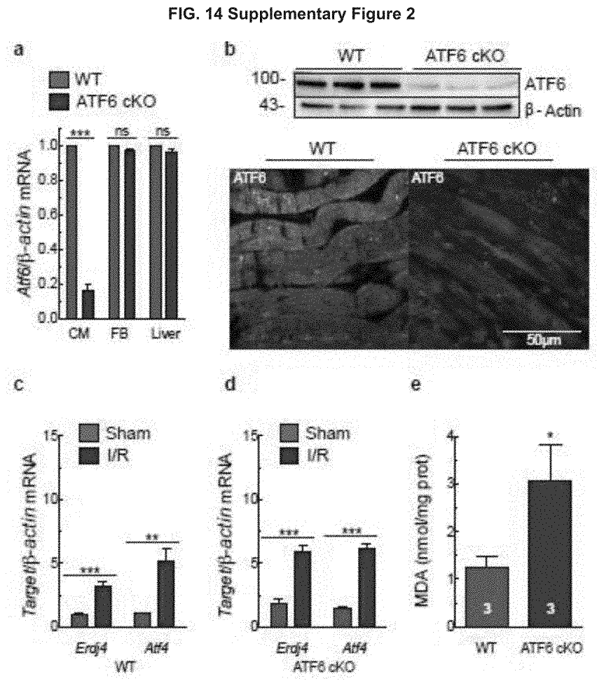

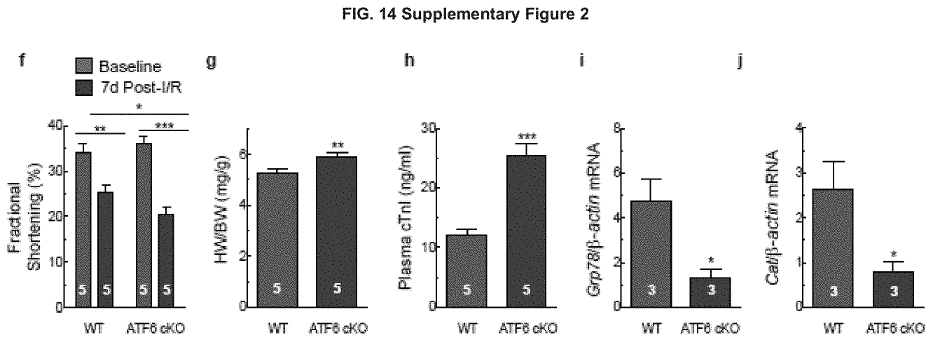

FIG. 7-12: Illustrate Exemplary Compounds Used in Methods as Provided Herein

FIG. 13 (or Supplementary FIG. 1): I/R Activates the UPR.

a, Immunoblots of neonatal rat ventricular myocytes (NRVM) for the proteins shown after I/R or tunicamycin (TM). b-d, Quantification of immunoblots from NRVM subjected to normoxia or I/R. ATF6, IRE1, and PERK activation are displayed as ratios of active fragment ATF6 (50 kd), spliced-XBP1 and phospho-PERK relative to ATF6 (90 kd), IRE1, and PERK, respectively. e, Immunocytofluorescence (ICF) for GRP78 or CAT (green), alpha-actinin (red) and nuclei (TOPRO-3) in isolated adult cardiomyocytes (AMVM) post-I/R. f, g, Quantification of immunoblots for Grp78 (f) or Cat (g) from NRVM subjected to normoxia or I/R. Data are represented as mean±s.e.m. *P<0.05, ***P<0.001.

FIG. 14 (or Supplementary FIG. 2): Endogenous ATF6 is Cardioprotective in a Model of a Chronic AMI.

a, qPCR for atf6 in isolated adult mouse ventricular myocytes (AMVM), isolated cardiac fibroblasts, or liver extracts from WT or ATF6 cKO mice. b, Immunoblot for Atf6 and loading control, β-actin, and IHC staining for ATF6 (cyan), tropomyosin (red), and nuclei (TOPRO-3) in LV of WT or ATF6 cKO mice. c, d, qPCR for IRE1 downstream target, Erdj4, or PERK downstream target, Atf4 in the border zone of WT (c) or ATF6 cKO (d) hearts 24-hours after I/R. e, Malondialdehyde (MDA) in WT and ATF6 cKO mice 24-hours post-I/R. f-j, Parameters from mice 7-days post f, Fractional shortening. Detailed analyses of echocardiography parameters are in Extended Data Table 1. g, Ratio of heart weight to body weight. h, Plasma cTnI. i, j, qPCR for Grp78 (i) or Cat (j) in border zone of mice. Data are represented as mean±s.e.m. *P<0.05, **P<0.01, ***P<0.001.

FIG. 15 (or Supplementary FIG. 3): Exemplary Compound 147 is Selectively Activates ATF6.

a, Immunoblots of UPR target proteins from NRVM 24-hours after treatment with compound 147 or tunicamycin (TM). b-f, Quantification of immunoblots of NRVM treated with control or compound 147. g, Immunoblot of NRVM infected with AdV encoding Flag-ATF6 (1-670) 24-hours after treatment with control or compound 147. Samples were performed in coordination with ChIP in FIG. 2e. h, Immunoblots of UPR target proteins from LV of WT or ATF6 cKO hearts 24-hours after treatment with control or compound 147. i, j, qPCR for Erdj4 or Atf4 in LV of WT (i) or ATF6 cKO (j) hearts 24-hours after treatment with control or compound 147. Data are represented as mean±s.e.m. *P<0.05, **P<0.01, ***P<0.001.

FIG. 16 (or Supplementary FIG. 4): Compound 147 Exhibits no Deleterious Effects, In Vivo.

a-c, qPCR for Erdj4 (a), Atf4 (b), and Atp2a2 (c) following experimental design in FIG. 4a d, Ratio of heart weight to body weight. e, Plasma cTnI. f, qPCR for cardiac pathology genes: Nppa (black), Nppb (red), Col1a2 (blue), and Myh7 (green) following experimental design in FIG. 4a. Data are represented as mean±s.e.m. ***P<0.001.

FIG. 17 (or Supplementary FIG. 5): Exemplary Compound 147 Decreases Pathological Remodeling 7 d Post-AMI.

a-b, qPCR for Erdj4 (a) or Atf4 (b) in border zone of mice from Trials 4-7 of the chronic I/R protocol shown in FIG. 5a. c, IHC staining for GRP78 or CAT (cyan), tropomyosin (red), and nuclei (TOPRO-3) in left ventricular free wall of sham hearts or the border zone of hearts from respective trials of experimental design in FIG. 5a. d, qPCR for cardiac pathology genes: Nppa (black), Nppb (red), Col1a2 (blue), and Myh7 (green) in border zone of mice from Trials 4-7 of the chronic I/R protocol shown in FIG. 5a. Statistics represent significance of entire gene sets for each trial from that of separate trials. e, IHC staining for cleaved caspase-3 (cyan), tropomyosin (red), and nuclei (TOPRO-3) in LV free wall of sham hearts or the border zone of hearts from indicated trials of experimental design in FIG. 5a. Data are represented as mean±s.e.m. *P<0.05, **P<0.01.

FIG. 18 (or Supplementary FIG. 6): Compound 147 is Protective in Multiple Models of Myocardial Damage.

a, Representative images of TTC-stained post-I/R hearts from Trials 8-10 of the acute I/R protocol shown in FIG. 6e. b, c, Relative infarct sizes (b) and plasma cTnI (c) of female mice 24-hours after reperfusion when following the acute I/R protocol shown in FIG. 6e. d, e, Relative infarct sizes (d) and plasma cTnI (e) of ATF6 cKO mice 24-hours post-I/R when following experimental Trials 8 (Con) and 9 (compound 147) of the acute I/R protocol. f, Experimental design for testing the effects of compound 147 in a different model of a AMI using isoproterenol. g-h, Relative infarct sizes (g), and plasma cTnI (h). Data are represented as mean±s.e.m. *P<0.05, **P<0.01, ***P<0.001.

Methods

Laboratory animals. The research reported in this article has been reviewed and approved by the San Diego State University Institutional Animal Care and Use Committee (IACUC), and conforms to the Guide for the Care and Use of Laboratory Animals published by the National Research Council. ATF6-floxed mice were a generous gift from Gokhan S. Hotamisligil. Briefly, ATF6-floxed mice were generated with a targeting construct flanking exons 8 and 9 of ATF6 with LoxP sequences on a C57B/6J background, as previously described²⁹. For preclinical efficacy testing of experimental compounds, wild-type (WT) 10-week old male or female C57B/6J mice were used (The Jackson Laboratory; Bar Harbor, Me.).

Patient samples. Human heart explants were obtained from ventricular myocardium of patients with advanced ischemic heart failure. Control patient ventricular explants were obtained from non-failing donor hearts deemed unsuitable for transplantation for non-cardiac reasons. Samples were collected as previously described³⁰. All study procedures were approved by the University of Pennsylvania Hospital Institutional Review Board.

Adeno-associated virus serotype 9 (AAV9). The plasmid encoding the human cardiac troponin T promoter driving Cre-recombinase was provided as a gift from Dr. Oliver Muller³¹. AAV9 preparation was carried out as previously described¹⁰. Non-anesthetized 8-week old ATF6-floxed mice were injected with 100 □L of AAV9-control or AAV9-cTnT-Cre containing 1×10¹¹ viral particles via the lateral tail vein using a 27-guage syringe and housed for 2 weeks before either sacrifice or experimental initiation.

Adenovirus. Construction of plasmid vectors encoding FLAG-tagged full length inactive ATF6 [ATF6(1-670)], TCR-α-HA, and empty vector (AdV-Con) has been previously described^{10,37}.

Cardiomyocyte isolation, culture and experimental design. Neonatal rat ventricular myocytes (NRVM) were isolated via enzymatic digestion, purified by Percoll density gradient centrifugation, and maintained in Dulbecco's modified Eagle's medium (DMEM)/F12 supplemented with 10% fetal bovine serum (FBS) and antibiotics (100 units/ml penicillin and 100 μg/ml streptomycin) on plastic culture plates that had been pre-treated with 5 μg/ml fibronectin, as previously described¹⁰. For all NRVM experiments, plating density was maintained at 4.5×10⁵ cells/well on 12-well plates. Adult mouse ventricular myocytes (AMVM) were isolated from WT or ATF6 cKO mice 24 hours after IV injection of control compound (2 mg/kg) or compound 147 (2 mg/kg). AMVM isolation was performed by cannulating the ascending aorta, followed by retroperfusion and collagenase digestion, as previously described¹⁰. For all experiments, AMVM were plated at a density of 5.0×10⁵ cells/well on 24-well plates that had been pre-treated with laminin (10 μg/ml) and incubated in maintaining medium (MEM medium, 1× insulin-transferrin-selenium, 10 mM HEPES, 1.2 mM CaCl₂ and 0.01% bovine serum albumin, 25 μM blebbistatin) for 16 hours before initiating experiments as previously described¹⁰. Sixteen hours after plating NRVM and AMVM were treated with control compound (10 μM), compound 147 (10 μM) or tunicamycin (10 μg/ml) for 24 hours in DMEM/F12 supplemented with bovine serum albumin (BSA) (1 mg/ml) for NRVM, or maintaining media for AMVM. For in vitro ischemia/reperfusion (I/R), ischemia was simulated by replacing all culture media with 0.5 ml of glucose-free DMEM containing 2% dialyzed FBS with either the control compound (10 μM), or compound 147 (10 μM), then incubated at 0.1% O₂ in a hypoxia chamber with an oxygen controller (ProOx P110™ oxygen controller, Biospherix, Parish, N.Y.) for 8 hours or 3 hours for NRVM or AMVM, respectively, as previously described¹⁰. Reperfusion was simulated by replacing culture media with DMEM/F12 supplemented with BSA (1 mg/ml) for NRVM or maintaining media for AMVM and incubating at 21% O₂ for an additional 24 hours. NRVM and AMVM reperfusion media were supplemented with control compound (10 μM), compound 147 (10 μM) throughout the duration of the reperfusion period. Viability was determined as numbers of calcein-AM-labeled NRVM or rod-shaped calcein-AM-labeled AMVM, using calcein-AM green (Thermo Fisher). Images were obtained with an IX70™ fluorescence microscope (Olympus, Melville, N.Y.). Numbers of viable, calcein-AM green-positive cells were counted using ImageJ or Image-Pro Plus software (Medium Cybernetics, Rockville, Md.).

Small interfering RNA (siRNA) transfection. Transfection of siRNA into NRVM was achieved using HiPerfect Transfection Reagent™ (Qiagen, Valencia, Calif.) following the vendor's protocol. Briefly, NRVM culture medium was replaced with DMEM/F12 supplemented with 0.5% FBS without antibiotics, 120 nM siRNA, and 1.25 μl HiPerfect/1 μl siRNA, then incubated for 16 hours, after which the culture medium was replaced with DMEM/F12 supplemented with BSA (1 mg/ml) for an additional 48 hours. The sequence of siRNA targeting rat ATF6 was 5-GCUCUCUUUGUUGUUGCUUAGUGGA-3 (SEQ ID NO:1), the sequence targeting rat catalase was 5-GGAACCCAAUAGGAGAUAAACUUAA-3 (SEQ ID NO:2) (cat #CatRSS302058, Stealth siRNA, Thermo Fisher), and the sequence targeting rat grp78 was 5-AGUGUUGGAAGAUUCUGA-3 (SEQ ID NO:3) (cat #4390771, Stealth siRNA, Thermo Fisher) as previously described¹⁰. A non-targeting sequence (cat #12935300, Thermo Fisher) was used as a control siRNA.

Immunoblot analysis. NRVM were lysed and subjected to immunoblot analysis, as previously described¹⁰. In brief, cultures were lysed with VC lysis buffer made from 20 mM Tris-HCl (pH 7.5), 150 mM NaCl, 0.1% SDS, 1% Triton X-100™, protease inhibitor cocktail (Roche Diagnostics, Indianapolis, Ind.) and phosphatase inhibitor cocktail (Roche Diagnostics). Samples comprising 10 μg of protein were mixed with Laemmli sample buffer, boiled, then subjected to SDS-PAGE followed by transfer onto PVDF membranes for immunoblotting. Full-length Atf6 (p90) was detected with an antibody from SAB Signalway™ Antibody (1:1000, cat #32008, College Park, Md.), while active Atf6 (p50) was detected with an antibody from Proteintech™ (1:1000, cat #24169-1-AP, Rosemont, Ill.). Other antibodies used include: anti-KDEL antibody (1:8,000, cat #ADI-SPA-827, Enzo Life Sciences, Farmingdale, N.Y.), which was used to detect GRP78™, anti-catalase (1:1000, cat #ab16731, Abcam), anti-IRE1 (1:500, cat #sc-390960, Santa Cruz), anti-XBP1s (1:1000, cat #619502, BioLegend™, San Diego, Calif.), anti-phospho-PERK (1:1000, cat #3179, Cell Signaling), anti-PERK (1:1000, cat #3192, Cell Signaling), anti-Anp (1:4000, cat #T-4014 , Peninsula), anti-Gapdh (1:25000, cat #G109a, Fitzgerald Industries International Inc.), HA-probe F-7 (Santa Cruz, SC-7392; 1:1,000) and anti-FLAG (1:3,000, cat #F1804, Sigma-Aldrich, St. Louis, Mo.). The oxidation state of ATF6 in NRVM treated with compound 147 was analyzed by gel-shift essentially as previously described³². Briefly, cells were lysed in low-stringency lysis buffer comprising 20 mM Tris-HCl (pH 7.5), 150 mM NaCl, 1% Triton X-100, protease inhibitor cocktail (Roche Diagnostics, Indianapolis, Ind.) and phosphatase inhibitor cocktail (Roche Diagnostics) and 20 μM 4-Acetamido-4′-Maleimidylstilbene-2,2′-Disulfonic Acid, Disodium Salt (AMS) (Thermo Fisher, cat #A485). AMS binds covalently to reduced thiols, typically on cysteine residues, and increases their molecular mass in SDS-PAGE. Thus, proteins that exhibit an upward shift when analyzed under non-reducing conditions compared to reducing are considered to have reduced thiols.

qPCR. Total RNA was extracted from left ventricular extract using the RNeasy Mini™ kit (Qiagen) as previously described¹⁰. All qPCR probes were obtained from Integrated DNA Technologies as previously described^{10,33}.

Immunocyto- and immunohistochemistry. NRVM and AMVM were plated on fibronectin and laminin-coated glass chamber slides, respectively as previously described¹⁰. In brief, cells were fixed with 4% paraformaldehyde, followed by permeabilization with 0.5% Triton-X. Adult mouse hearts were paraffin-embedded after fixation in neutral buffered 10% formalin via abdominal aorta retroperfusion as previously described¹⁰. The infarct border zone was imaged in hearts subjected to surgical I/R. The infarct border zone was identified as an area that stained positively for the cardiac muscle protein, tropomyosin that was adjacent to an area that did not stain for tropomysin (infarct zone) due to the absence of viable myocytes. The left ventricular free wall was imaged in sham and non-injured hearts. Primary antibodies used were anti-α-actinin (1:200, cat #A7811, Sigma-Aldrich), anti-tropomyosin (1:200, cat #T9283, Sigma-Aldrich), anti-GRP78 (C-20, 1:30, cat #SC-1051, Santa Cruz), anti-catalase (1:100, Abcam), anti-ATF6 (targeting to N-terminus of ATF6, 1:50, cat #sc-14250, Santa Cruz), and anti-cleaved caspase-3 (1:100, cat #D175, Cell Signaling). Slides were incubated with appropriate fluorophore-conjugated secondary antibodies (1:100, Jackson ImmunoResearch Laboratories, West Grove, Pa.) followed by nuclei counter stain Topro-3 (1:2000, Thermo Fisher). Images were obtained using laser scanning confocal microscopy on an LSM 710 confocal laser scanning microscope (Carl Zeiss, Oberkochen, Germany).

ERAD Assay. ER-associated degradation (ERAD) was determined using a C-terminal HA-tagged version of the model chronic misfolded substrate, TCR-α-HA as previously described³⁷.

Luciferase Secretion Assay. NRVM were cotransfected with pcDNA plasmid as well as p-SV-β-galactosidase control vector and pCMV-GLuc plasmid (NEB, N8081S) using FuGENE6 (2 μg cDNA, 2:1, FuGENE:cDNA) essentially as previously described³⁴.

Chromatin immunoprecipitation (ChIP). ChIP assays were performed essentially as previously described¹⁰. Briefly, AdV-FLAG-ATF6(1-670) infected NRVM were treated with fixing buffer (50 mM HEPES-KOH, pH 7.5, 100 mM NaCl, 1 mM EDTA, 0.5 mM EGTA, and 1% formaldehyde) for 10 min, quenched with 125 mM glycine, and scraped into ice-cold PBS. Cells were centrifuged, resuspended in lysis buffer (50 mM HEPES, pH 7.9, 140 mM NaCl, 1 mM EDTA, 10% glycerol, 0.5% NP-40, 0.25% Triton X-100, and protease inhibitor cocktail), and incubated on ice for 10 min. After centrifugation at 1,800×g for 10 min, the pellets were washed with buffer containing 10 mM Tris, pH 8.1, 200 mM NaCl, 1 mM EDTA, and 0.5 mM EGTA, resuspended in shearing buffer (0.1% SDS, 1 mM EDTA, and 10 mM Tris, pH 8.1), and then transferred to microTUBEs (Covaris, Woburn, Mass.). Chromatin was sheared by sonication for 15 min using an M220 focused ultrasonicator (Covaris). Triton X-100 and NaCl were added to the final concentration of 1% Triton and 150 mM NaCl followed by centrifugation at 16,000×g for 10 min. Immunoprecipitation was performed by incubated 140 μl of sheared chromatin with 5 μg of anti-FLAG antibody (cat #F1804, Sigma-Aldrich) and 260 μl of immunoprecipitation buffer (0.1% SDS, 1 mM EDTA, 10 mM Tris, pH 8.1, 1% Triton X-100, and 150 mM NaCl) at 4° C. overnight. Protein A/G magnetic beads (5 BcMag, Bioclone, San Diego, Calif.) were added to the mixtures and incubated at 4° C. for 1.5 h. Magnetic beads were sequentially washed with low salt wash buffer (0.1% SDS, 1% Triton X-100, 2 mM EDTA, 20 mM HEPES-KOH, pH7.9, and 150 mM NaCl), high salt wash buffer with 500 mM NaCl, LiCl wash buffer (100 mM Tris-HCl, pH 7.5, 0.5 M LiCl, 1% NP-40, and 1% deoxycholate acid), and TE buffer (10 mM Tris-HCl, pH 8.0 and 0.1 mM EDTA). Immune complexes were eluted by incubating beads with proteinase K digestion buffer (20 mM HEPES, pH 7.9, 1 mM EDTA, 0.5% SDS, and 0.4 mg/ml proteinase K) at 50° C. for 15 min. Formaldehyde crosslinking was reversed by incubating with 0.3 M NaCl and 0.3 mg/ml RNase A at 65° C. overnight. Samples were further incubated with 550 μg/ml proteinase K at 50° C. for 1 h. DNA was purified using NucleoSpin Gel™ and PCR Clean-up Kit™ (Macherey-Nagel, Bethlehem, Pa.) and eluted by 30 μl of water. Two μl of DNA was used for qRT-PCR analysis with primers targeting rat Hspa5 (5′-GGTGGCATGAACCAACCAG-3′ (SEQ ID NO:4) and 5′-GCTTATATATCCTCCCCGC-3′) (SEQ ID NO:5), rat Cat ERSE-1 (5′-CTACCCACCAATTAGTACCAAATAA-3′ (SEQ ID NO:6) and 5′-AGAAGGGACAGGATTGGAAG-3′) (SEQ ID NO:7), rat Cat ERSE-2 (5′-CACATTCTAGGGACAGTGTAGATG-3′ (SEQ ID NO:8) and 5′-ACCTTGATTATGGGCTGTGG-3′) (SEQ ID NO:9), rat Pdia6 ERSE (5′-CACATGAGCGAAATCCACAGA-3′ (SEQ ID NO:10) and 5′-ACTAGTCGAGCCATGCTGAT-3′) (SEQ ID NO:11), rat HO-1 (5′-GGGCTACTCCCGTCTTCCTG-3′ (SEQ ID NO:12) and 5′-CCTTTCCAGAACCCTCTACTCTACTC-3′) (SEQ ID NO:13), or rat Gapdh (5′-ATGCGGTTTCTAGGTTCACG-3′ (SEQ ID NO:14) and 5′-ATGTTTTCTGGGGTGCAAAG-3′) (SEQ ID NO:15). Pdia6 served as a positive control for a known ATF6 target gene in cardiac myocytes while HO-1 and Gapdh served as negative controls. ChIP signals obtained from the qRT-PCR were normalized to the input DNA.

Ex vivo ischemia/reperfusion. Hearts from WT or ATF6 cKO mice that had previously received 2 mg/kg IV administration of control compound or exemplary compound 147 were rapidly excised and cannulated via the ascending aorta and subjected to global I/R, as previously described³⁵. Here, the hearts were subjected to 20 minutes global no-flow ischemia followed by reperfusion for 1 hour. Left ventricular developed pressure (LVDP) was measured using a pressure sensor balloon placed into the left ventricle and analyzed using Powerlab™ software (ADInstruments, Colorado Springs, Colo.).

In vivo myocardial ischemia/reperfusion. Surgical myocardial I/R was performed as previously described¹⁰. Briefly, mice were anesthetized with 2% isoflurane and a thoracotomy was performed to isolate the heart, after which the left anterior descending coronary artery (LAD) was ligated with a 6-0 Prolene™ suture for 30 minutes, followed by suture removal and either 24 hours or 7 days of reperfusion. Regional ischemia was confirmed by visual inspection of the discoloration of the myocardium distal of the ligation, which is characteristic of impaired blood flow. Animals assigned as shams underwent the thoracotomy surgical procedure, but weren't subjected to LAD ligation. Animals were randomly assigned to trial groups prior to outset of the experiment by a single investigator, while the surgeon and data analyst were blinded to trial assignments. Animals designated to receive either control compound or compound 147 at the time of reperfusion received 2 mg/kg of respective compounds via IV injection 5 minutes prior to release of the ligation. Twenty-four hours after reperfusion, 1% of Evans Blue was injected apically to determine the area at risk (AAR). Hearts were harvested and 1-mm sections of the hearts were stained with 1% 2,3,5-triphenyltetrazolium chloride (TTC) to measure the infarcted area (INF) as previously described³⁶. The AAR, INF and left ventricle area (LV) from digitized images of heart sections were analyzed using ImageJ software. For all infarct data presented, respective AAR was normalized to total LV area and all compared trials displayed the same AAR/LV ratios. A separate investigator analyzed the AAR, INF, and LV and was blinded to the animal trial assignments. Just prior to sacrifice, post-I/R, animals were anesthetized and 0.5 mL of arterial blood were obtained via inferior vena cava puncture as previously described³³. Blood was placed in heparin- and EDTA-coated vacutainer (BD Vacutainer) and centrifuged at 3000 rpm for 10 minutes and plasma samples were analyzed for cardiac troponin I with a Mouse cTnI High-Sensitivity ELISA kit (Life Diagnostics, Inc.).

In vivo renal ischemia/reperfusion. Surgical renal I/R was performed as previously described³⁷. Briefly, mice were anesthetized with 2% isoflurane and a 3 cm incision was made upon the abdominal midline and the abdominal cavity entered via an incision along the linea alba. The right kidney was visualized and separated from surrounding connective tissue. The right ureter and right renal portal system was permanently ligated and a right unilateral nephrectomy performed. Subsequently, the left kidney was visualized and separated from surrounding connective tissue. A Bulldog Clamp (Fine Science Tools, Foster City, Calif.) was applied temporarily ligating the left renal portal system for a period of 30 minutes. Global ischemia was confirmed by visual inspection of the discoloration of the kidney of the ligation, which is characteristic of impaired blood flow. After that duration, the Bulldog Clamp was removed and the abdomen closed with instant tissue adhesive. Animals were randomly assigned to trial groups prior to outset of the experiment by a single investigator, while the data analyst was blinded to trial assignments. Animals designated to receive either control compound or compound 147 at the time of reperfusion received 2 mg/kg of respective compounds via IV injection 5 minutes prior to release of the ligation. Twenty-four hours after reperfusion, kidneys were harvested and 1-mm sections of the kidneys were stained with 1% TTC to measure the infarcted area (INF) as previously described³⁶. Just prior to sacrifice, post-I/R, animals were anesthetized and 0.5 mL of arterial blood were obtained via inferior vena cava puncture as previously described³³. Blood was placed in heparin- and EDTA-coated vacutainer (BD Vacutainer) and centrifuged at 3000 rpm for 10 minutes and plasma samples were analyzed for creatinine as a measure of glomerular filtration rate and renal functional output with a Creatinine Assay kit (Abcam).

In vivo cerebral ischemia/reperfusion. Surgical cerebral I/R was performed as previously described¹¹. Briefly, mice were anesthetized with 2% isoflurane and a 3 cm incision was made along the midline of the ventral surface of the neck along the left side of the trachea. The left external and internal carotid arteries were visualized and dissected from surrounding connective tissue without disturbing tangential nerves. An 8-0 catheter filament 10 mm in length (Doccol Corporation) was be inserted into the middle cerebral artery (MCA) via the internal carotid artery. This occluded blood flow to the MCA and was left in position for a period of 30 minutes. After that duration, the catheter was removed and the neck closed with instant tissue adhesive. Animals were randomly assigned to trial groups prior to outset of the experiment by a single investigator, while the data analyst was blinded to trial assignments. Animals designated to receive either control compound or compound 147 at the time of reperfusion received 2 mg/kg of respective compounds via IV injection 5 minutes prior to release of the ligation. Twenty-four hours after reperfusion, brains were harvested and 1-mm sections of the brains were stained with 1% TTC to measure the infarcted area (INF) as previously described³⁶. Just prior to sacrifice animals were assigned a behavioral score to assess the severity of neurological function and deficit as a result of the cerebral ischemia. The scoring was performed based on the Bederson Neurological Examination Grading System³⁸, where a grade of 0 corresponded to a normal function with no observable deficit, grade 1 to a moderate deficit with animals exhibiting forearm flexion, grade 2 to a severe deficit with decreased resistance to a lateral push when suspended by the tail and lethargy, and grade 3 to a severe deficit with extreme lethargy and circling behavior in the cage.

Hepatic triglyceride assay. Hepatic triglyceride assay was performed as previously described³⁹. Briefly, livers were harvested and 10 mg extracts were homogenized and analyzed for triglyceride content using the EnzyChrom Triglyceride Assay Kit™ (BioAssay Systems).

Transthoracic echocardiography. Transthoracic echocardiography was performed using an ultrasound imaging system (Vevo 2100 System™, Fujifilm VisualSonics, Toronto, Ontario, Canada) as described⁴⁰. Diastolic function was determined as previously described³³. Briefly, echocardiography coupled with pulse-wave Doppler was used to visualize transmitral flow velocities and were recorded by imaging the mitral orifice at the point of the mitral leaflets. Waveforms were recorded and analyzed for peak early- and late-diastolic transmitral flow velocities corresponding to E and A waves, respectively.

Acute isoproterenol myocardial damage. Myocardial damage was induced by administering high-dose (200 mg/kg) isoproterenol via intraperitoneal injection in mice as previously described³³.

Malondialdehyde assay. Lipid peroxidation was determined by measuring the levels of malondialdehyde (MDA) using a TBARS assay kit (Cayman Chemical, Ann Arbor, Mich.) according to the manufacturer's instructions as previously described¹⁰.

In vivo experimental compound administration. Control compound and compound 147 were suspended to a final concentration of 0.2 mg/mL in 10% DMSO. Mice were weighed prior to administration of compounds and, subsequently, non-anesthetized 10-week old WT or ATF6 cKO mice were injected with ˜250 μL of stock compounds via the lateral tail vein depending upon body mass to ensure accurate administration of 2 mg/kg. This dose was established in preliminary experiments with the control compound or compound 147 where it was shown to activate Atf6 in vivo; the prototypical UPR inducer, tunicamycin, which was also administered to mice at 2 mg/kg, as previously shown⁴¹ was used as a control. Since compound 147 and tunicamycin have similar molecular weights, this dose of compound 147 is near the molar equivalent of the typical dose of tunicamycin. It is relevant to note that for compound 147, a dose of 2 mg/kg is similar to FDA-approved cardiovascular drugs, such as many angiotensin-converting enzyme (ACE) inhibitors, which are used in small-animal models at 2 mg/kg⁴².

Statistics. For studies involving induction of myocardial damage, either through surgical I/R or isoproterenol administration, cohort sizes were based on a predictive power analysis to achieve 5% error and 80% power. All acute in vivo I/R studies in which compound 147 was administered in preclinical trial design were conducted such that the surgeon and data analyst was blinded to the group assignments. Two-group comparisons were performed using Student's two-tailed t-test, and all multiple group comparisons were performed using a one-way ANOVA with a Newman-Keuls post-hoc analysis. Data are represented as mean with all error bars indicating±s.e.m. *P<0.05, **P<0.01, ***P<0.001.

### REFERENCES

1 Hartl, F. U., Bracher, A. & Hayer-Hartl, M. Molecular chaperones in protein folding and proteostasis. Nature 475, 324-332, doi:10.1038/nature10317 (2011).
2 Labbadia, J. & Morimoto, R. I. The biology of proteostasis in aging and disease. Annu Rev Biochem 84, 435-464, doi:10.1146/annurev-biochem-060614-033955 (2015).
3 Das, I. et al. Preventing proteostasis diseases by selective inhibition of a phosphatase regulatory subunit. Science 348, 239-242, doi:10.1126/science.aaa4484 (2015).
4 Roth, D. M. & Balch, W. E. Modeling general proteostasis: proteome balance in health and disease. Curr Opin Cell Biol 23, 126-134, doi:10.1016/j.ceb.2010.11.001 (2011).
5 Walter, P. & Ron, D. The unfolded protein response: from stress pathway to homeostatic regulation. Science 334, 1081-1086, doi:10.1126/science.1209038 (2011).
6 Smith, M. H., Ploegh, H. L. & Weissman, J. S. Road to ruin: targeting proteins for degradation in the endoplasmic reticulum. Science 334, 1086-1090, doi:10.1126/science.1209235 (2011).
7 Chiang, W. C., Hiramatsu, N., Messah, C., Kroeger, H. & Lin, J. H. Selective activation of ATF6 and PERK endoplasmic reticulum stress signaling pathways prevent mutant rhodopsin accumulation. Invest Ophthalmol Vis Sci 53, 7159-7166, doi:10.1167/iovs.12-10222 (2012).
8 Cooley, C. B. et al. Unfolded protein response activation reduces secretion and extracellular aggregation of amyloidogenic immunoglobulin light chain. Proc Natl Acad Sci USA 111, 13046-13051, doi:10.1073/pnas.1406050111 (2014).
9 Martindale, J. J. et al. Endoplasmic reticulum stress gene induction and protection from ischemia/reperfusion injury in the hearts of transgenic mice with a tamoxifen-regulated form of ATF6. Circ Res 98, 1186-1193, doi:01.RES.0000220643.65941.8d [pii] 10.1161/01.RES.0000220643.65941.8d (2006).
10 Jin, J. K. et al. ATF6 Decreases Myocardial Ischemia/Reperfusion Damage and Links ER Stress and Oxidative Stress Signaling Pathways in the Heart. Circ Res 120, 862-875, doi:10.1161/CIRCRESAHA.116.310266 (2017).
11 Yu, Z. et al. Activation of the ATF6 branch of the unfolded protein response in neurons improves stroke outcome. J Cereb Blood Flow Metab 37, 1069-1079, doi:10.1177/0271678X16650218 (2017).
12 Roth, G. A. et al. Global, Regional, and National Burden of Cardiovascular Diseases for 10 Causes, 1990 to 2015. J Am Coll Cardiol 70, 1-25, doi:10.1016/j.jacc.2017.04.052 (2017).
13 Frangogiannis, N. G. Pathophysiology of Myocardial Infarction. Compr Physiol 5, 1841-1875, doi:10.1002/cphy.c150006 (2015).
14 Hausenloy, D. J. & Yellon, D. M. Ischaemic conditioning and reperfusion injury. Nat Rev Cardiol 13, 193-209, doi:10.1038/nrcardio.2016.5 (2016).
15 Bulluck, H., Yellon, D. M. & Hausenloy, D. J. Reducing myocardial infarct size: challenges and future opportunities. Heart 102, 341-348, doi:10.1136/heartjnl-2015-307855 (2016).
16 Kalogeris, T., Baines, C. P., Krenz, M. & Korthuis, R. J. Ischemia/Reperfusion. Compr Physiol 7, 113-170, doi:10.1002/cphy.c160006 (2016).
17 Murphy, E. & Steenbergen, C. Mechanisms underlying acute protection from cardiac ischemia-reperfusion injury. Physiol Rev 88, 581-609, doi:10.1152/physrev.00024.2007 (2008).
18 Yellon, D. M. & Hausenloy, D. J. Myocardial reperfusion injury. N Engl J Med 357, 1121-1135, doi:10.1056/NEJMra071667 (2007).
19 Wang, J., Lee, J., Liem, D. & Ping, P. HSPAS Gene encoding Hsp70 chaperone BiP in the endoplasmic reticulum. Gene 618, 14-23, doi:10.1016/j.gene.2017.03.005 (2017).
20 Jin, J. K. et al. ATF6 Decreases Myocardial Ischemia/Reperfusion Damage and Links ER Stress and Oxidative Stress Signaling Pathways in the Heart. Circ Res, doi:10.1161/CIRCRESAHA.116.310266 (2016).
21 Kumar, M. et al. Animal models of myocardial infarction: Mainstay in clinical translation. Regul Toxicol Pharmacol 76, 221-230, doi:10.1016/j.yrtph.2016.03.005 (2016).
22 Dixon, J. A. & Spinale, F. G. Pathophysiology of myocardial injury and remodeling: implications for molecular imaging. J Nucl Med 51 Suppl 1, 102S-106S, doi:10.2967/jnumed.109.068213 (2010).
23 Plate, L. et al. Small molecule proteostasis regulators that reprogram the ER to reduce extracellular protein aggregation. Elife 5, doi:10.7554/eLife.15550 (2016).
24 Thuerauf, D. J. et al. Sarco/endoplasmic reticulum calcium ATPase-2 expression is regulated by ATF6 during the endoplasmic reticulum stress response: intracellular signaling of calcium stress in a cardiac myocyte model system. J Biol Chem 276, 48309-48317, doi:10.1074/jbc.M107146200 M107146200 [pii] (2001).
25 Gwathmey, J. K., Yerevanian, A. & Hajjar, R. J. Targeting sarcoplasmic reticulum calcium ATPase by gene therapy. Hum Gene Ther 24, 937-947, doi:10.1089/hum.2013.2512 (2013).
26 Kroeger, H. et al. The unfolded protein response regulator ATF6 promotes mesodermal differentiation. Sci Signal 11, doi:10.1126/scisignal.aan5785 (2018).
27 Ron, D. & Walter, P. Signal integration in the endoplasmic reticulum unfolded protein response. Nat Rev Mol Cell Biol 8, 519-529, doi:10.1038/nrm2199 (2007).
28 Martinez, G., Duran-Aniotz, C., Cabral-Miranda, F., Vivar, J. P. & Hetz, C. Endoplasmic reticulum proteostasis impairment in aging. Aging Cell 16, 615-623, doi:10.1111/ace1.12599 (2017).
29 Engin, F. et al. Restoration of the unfolded protein response in pancreatic beta cells protects mice against type 1 diabetes. Sci Transl Med 5, 211ra156, doi:10.1126/scitranslmed.3006534 (2013).
30 Bedi, K. C., Jr. et al. Evidence for Intramyocardial Disruption of Lipid Metabolism and Increased Myocardial Ketone Utilization in Advanced Human Heart Failure. Circulation 133, 706-716, doi:10.1161/CIRCULATIONAHA.115.017545 (2016).
31 Werfel, S. et al. Rapid and highly efficient inducible cardiac gene knockout in adult mice using AAV-mediated expression of Cre recombinase. Cardiovasc Res 104, 15-23, doi:10.1093/cvr/cvu174 (2014).
32 Nadanaka, S., Okada, T., Yoshida, H. & Mori, K. Role of disulfide bridges formed in the luminal domain of ATF6 in sensing endoplasmic reticulum stress. Mol Cell Biol 27, 1027-1043, doi:10.1128/MCB.00408-06 (2007).
33 Wallner, M. et al. Acute Catecholamine Exposure Causes Reversible Myocyte Injury Without Cardiac Regeneration. Circ Res 119, 865-879, doi:10.1161/CIRCRESAHA.116.308687 (2016).
34 Lynch, J. M. et al. A thrombospondin-dependent pathway for a protective ER stress response. Cell 149, 1257-1268, doi:S0092-8674(12)00572-7 [pii] 10.1016/j.ce11.2012.03.050 (2012).
35 Jin, J. K. et al. Localization of phosphorylated alphaB-crystallin to heart mitochondria during ischemia-reperfusion. Am J Physiol Heart Circ Physiol 294, H337-344, doi:00881.2007 [pii] 10.1152/ajpheart.00881.2007 (2008).
36 Xie, M. et al. Histone deacetylase inhibition blunts ischemia/reperfusion injury by inducing cardiomyocyte autophagy. Circulation 129, 1139-1151, doi:10.1161/CIRCULATIONAHA.113.002416 (2014).
37 Wei, Q. & Dong, Z. Mouse model of ischemic acute kidney injury: technical notes and tricks. Am J Physiol Renal Physiol 303, F1487-1494, doi:10.1152/ajprenal.00352.2012 (2012).
38 Bederson, J. B. et al. Rat middle cerebral artery occlusion: evaluation of the model and development of a neurologic examination. Stroke 17, 472-476 (1986).
39 DeZwaan-McCabe, D. et al. ER Stress Inhibits Liver Fatty Acid Oxidation while Unmitigated Stress Leads to Anorexia-Induced Lipolysis and Both Liver and Kidney Steatosis. Cell Rep 19, 1794-1806, doi:10.1016/j.celrep.2017.05.020 (2017).
40 Doroudgar, S. et al. Hrd1 and ER-Associated Protein Degradation, ERAD, are Critical Elements of the Adaptive ER Stress Response in Cardiac Myocytes. Circ Res 117, 536-546, doi:10.1161/CIRCRESAHA.115.306993 (2015).
41 Wu, J. et al. ATF6alpha optimizes long-term endoplasmic reticulum function to protect cells from chronic stress. Dev Cell 13, 351-364, doi:10.1016/j.devce1.2007.07.005 (2007).
42 Eckman, E. A. et al. Regulation of steady-state beta-amyloid levels in the brain by neprilysin and endothelin-converting enzyme but not angiotensin-converting enzyme. J Biol Chem 281, 30471-30478, doi:10.1074/jbc.M605827200 (2006).

A number of embodiments of the invention have been described. Nevertheless, it can be understood that various modifications may be made without departing from the spirit and scope of the invention. Accordingly, other embodiments are within the scope of the following claims.

1. A method for:
selectively inducing only the ATF6 arm of the unfolded protein response (UPR) in a cell, a tissue or in a mammal, wherein optionally the mammal is a human,
mitigating, ameliorating, treating or preventing a proteostasis-based injury or dysfunction in a tissue or organ,
protecting a mammalian heart or a mammalian tissue from an acute or a long term ischemia/reperfusion (I/R) injury or damage,
pharmacologically activating ATF6 or the ATF6 arm of the unfolded protein response (UPR) in a cell or in vivo,
ameliorating, preventing or treating the loss of cardiac myocytes during ischemia/reperfusion (I/R) injury or damage,
ameliorating, preventing or treating ischemic heart disease in an individual in need thereof,
ameliorating, preventing or treating acute myocardial infarction (AMI) or tissue loss or damage occurring as a result of the AMI in an individual in need thereof, and/or
ameliorating, preventing or treating an amyloid-based disease, optionally amyloidosis, or an amyloid-based or amyloid-related neurodegenerative disease,
comprising:
administering to the cell, the tissue, the mammal or the individual in need thereof:
(a) (i) a compound have a structure a set forth in Formula I: 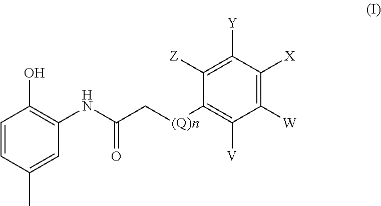
wherein Q is S, O, CH₂, CHF, or CF₂, n=1, 2, 3, or 4, when Q is CH₂, CHF, or CF₂; n is 1 when Q is S or O, and V, W, X, Y and Z are each independently hydrogen, halogen, alkyl, alkenyl, alkynyl, or alkoxy; or a pharmaceutically acceptable salt thereof,
(ii) a pharmaceutically acceptable salt or solvate, an optical isomer, or a racemic mixture or enantiomer of a compound of (i),
(iii) a compound as set forth in WO2017/117430 A1, or a pharmaceutically acceptable salt or solvate, optical isomer, or racemic mixture or enantiomer thereof;
(iv) a compound as set forth in FIGS. 7 to 12, or a pharmaceutically acceptable salt or solvate, optical isomer, or racemic mixture or enantiomer thereof; or,
(v) any mixture of compounds of (i) to (v); or
(b) a pharmaceutical composition or formulation comprising a compound of (a), or comprising at least one compound of (a),
thereby:
selectively inducing only the ATF6 arm of the unfolded protein response (UPR) in a cell, a tissue or in a mammal, wherein optionally the mammal is a human,
mitigating, ameliorating, treating or preventing a proteostasis-based injury or dysfunction,
protecting a mammalian heart, kidney, liver or brain, or a mammalian tissue from an acute or a long term ischemia/reperfusion (I/R) injury or damage,
pharmacologically activating ATF6 or the ATF6 arm of the unfolded protein response (UPR) in a cell or in vivo,
ameliorating, preventing or treating the loss of cardiac myocytes during ischemia/reperfusion (I/R) injury or damage,
ameliorating, preventing or treating ischemic heart disease in an individual in need thereof,
ameliorating, preventing or treating acute myocardial infarction (AMI) or tissue loss or damage occurring as a result of the AMI in an individual in need thereof, and/or
ameliorating, preventing or treating an amyloid-based disease, optionally amyloidosis, or an amyloid-based or amyloid-related neurodegenerative disease.

2. The method of claim 1, wherein the compound, pharmaceutical composition or formulation is administered in the form of an implant or a stent.

3. The method of claim 1, wherein the compound, pharmaceutical composition or formulation is suitable for or is formulated for: topical, oral, parenteral, intrathecal or intravenous infusion administration.

4. The method of claim 1, wherein the compound, pharmaceutical composition or formulation is suitable for or is formulated for: human or veterinary administration, wherein optionally said composition is suitable for or formulated for administration to a domestic, zoo, laboratory or farm animal, and optionally the animal is a dog or a cat.

5. The method of claim 1, wherein the compound, pharmaceutical composition or formulation is administered in a pharmaceutically effective dosage or amount, and optionally the pharmaceutically effective dosage or amount is or total daily dosage is between about 0.5 mg and about 5000 mg.

6. A product of manufacture comprising or having contained therein a compound, pharmaceutical composition or formulation of claim 1(a) or 1(b), wherein optionally the product of manufacture is an implant or a stent.

7-8. (canceled)

9. The method of claim 1, wherein the compound having a structure a set forth in Formula I is compound 147:

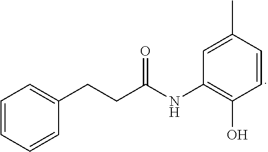 [SMILES: Cc1ccc(O)c(NC(=O)CCc2ccccc2)c1]

10. The method of claim 1, wherein the pharmaceutical composition or formulation further comprises a pharmaceutically acceptable excipient.

11. The method of claim 1, wherein the proteostasis-based injury or dysfunction comprises an ischemia/reperfusion (I/R) injury or damage in a heart, kidney, liver, muscle, central nervous system or brain.

12. The method of claim 1, wherein the proteostasis-based injury or dysfunction comprises a dysregulated proteostasis in the liver.

13. The method of claim 1, wherein the tissue or organ is a human tissue or organ.

14. The method of claim 1, wherein the method comprises protecting a mammalian heart or a mammalian brain, a kidney or a liver tissue from an acute or a long term ischemia/reperfusion (I/R) injury or damage.

15. The method of claim 1, wherein the amyloid-based disease is amyloidosis, or an amyloid-based or amyloid-related neurodegenerative disease.

16. The method of claim 15, wherein the amyloid-based or amyloid-related neurodegenerative disease is a central nervous system (CNS) or peripheral nervous system (PNS) neurodegenerative disease, or Alzheimer's disease.

17. The method of claim 2, wherein the implant or stent has contained therein or carries, releases or delivers the compound, pharmaceutical composition or formulation, thereby delivering or contacting the compound, pharmaceutical composition or formulation to or with the cell, the tissue, the mammal or the individual in need thereof.

18. The method of claim 1, wherein the compound, pharmaceutical composition or formulation is suitable for (or formulated for) administration as a (or in the form of a) patch, adhesive tape, gel, liquid or suspension, powder, spray, aerosol, lyophilate, lozenge, pill, geltab, tablet, capsule, stent and/or implant.

19. The method of claim 5, wherein the compound, pharmaceutical composition or formulation is administered in a pharmaceutically effective dosage or amount, or the pharmaceutically effective dosage or amount is, or total daily dosage is: between about 1 mg and about 1000 mg; or is between about 5 mg and about 500 mg, 10 mg and about 400 mg, 20 mg and about 250 mg; or is about 5 mg and about 150 mg; or is between about 1 mg and about 75 mg; or is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, or about 75 mg.

20. The method of claim 5, wherein the pharmaceutically effective dosage or amount is administered daily, twice a day (bid), three times a day (tid) or four or more times a day.
